(19) United States
(12) Patent Application Publication
(10) Pub. No.: US 2024/0374600 A1
(43) Pub. Date: Nov. 14, 2024

(54) METHODS AND COMPOSITIONS FOR TREATING CANCER

(71) Applicant: Onconova Therapeutics, Inc., Newtown, PA (US)

(72) Inventors: Steven M. Fruchtman, New York, NY (US); Matthew Parris, Plainsboro, NJ (US); Mark S. Gelder, Montgomery, TX (US)

(21) Appl. No.: 18/664,554

(22) Filed: May 15, 2024

Related U.S. Application Data
(63) Continuation of application No. PCT/US22/80194, filed on Nov. 18, 2022.
(60) Provisional application No. 63/280,948, filed on Nov. 18, 2021.

(51) Int. Cl. A61K 31/519; A61K 9/00; A61K 31/4196; A61K 31/573; A61K 45/06

(52) U.S. Cl. CPC A61K 31/519; A61K 9/0053; A61K 31/4196; A61K 31/573; A61K 45/06

(57) ABSTRACT

Disclosed herein is a method of treating endometrial cancer by administering to the subject a solid pharmaceutical composition comprising 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, alone or in combination with a second agent such as letrozole.

### CROSS-REFERENCE

This application is a continuation of International Application No. PCT/US22/80194 filed Nov. 18, 2022, which claims the benefit of U.S. Provisional Application No. 63/280,948, filed Nov. 18, 2021, each of which is entirely incorporated by reference herein.

### BACKGROUND

Endometrial cancer is the most common gynecologic malignancy. A large subset of endometrial cancers have alterations in both the phosphatidylinositol-4,5-bisphosphate 3-kinase (PI3K) and mitogen-activated protein kinase (MAPK) pathways.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY OF THE INVENTION

In some embodiments, the present disclosure provides a method of treating hormone receptor positive endometrial cancer in a subject in need thereof, the method comprising administering to the subject a therapeutically-effective amount of a compound of formula (I)

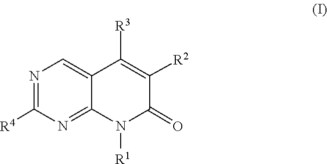

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof.

In some embodiments, the present disclosure provides a method of treating endometrial cancer in a subject in need thereof, the method comprising administering to the subject a therapeutically-effective amount of a compound of formula (I)

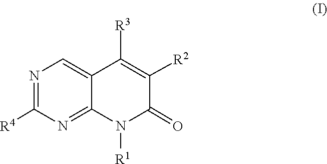

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof.

In some embodiments, the present disclosure provides a method of treating endometrial cancer, e.g., hormone receptor positive endometrial cancer, estrogen receptor positive endometrial cancer, progesterone receptor positive endometrial cancer, estrogen receptor positive, progesterone receptor positive endometrial cancer, in a subject in need thereof, the method comprising administering to the subject a therapeutically-effective amount of a compound of formula (I)

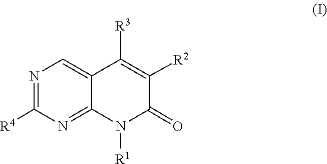

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the subject received a therapy other than the compound for the endometrial cancer, e.g., hormone receptor positive endometrial cancer, estrogen receptor positive endometrial cancer, progesterone receptor positive endometrial cancer, estrogen receptor positive, progesterone receptor positive endometrial cancer prior to the administering

In some embodiments, the present disclosure provides a method of treating endometrial cancer, e.g., hormone receptor positive endometrial cancer, estrogen receptor positive endometrial cancer, progesterone receptor positive endometrial cancer, estrogen receptor positive, progesterone receptor positive endometrial cancer in a subject in need thereof, the method comprising administering to the subject a therapeutically-effective amount of a compound of formula (I)

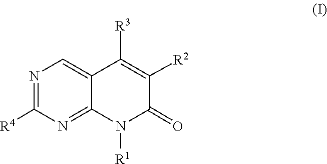

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof;
wherein the administering is once daily for at least 4 weeks.

In some embodiments, the present disclosure provides a method of treating endometrial cancer, e.g., hormone receptor positive endometrial cancer, estrogen receptor positive endometrial cancer, progesterone receptor positive endometrial cancer, estrogen receptor positive, progesterone receptor positive endometrial cancer in a subject in need thereof, the method comprising administering to the subject a therapeutically-effective amount of a compound of formula (I)

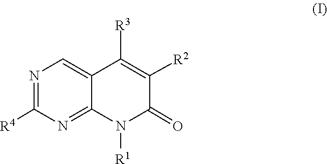

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃, or a pharmaceutically-acceptable salt thereof;
wherein the administering is a 4-week cycle of:
   (i) a continuous, three-week period of once-daily administration; and
   ii) immediately following the three-week period, one week of no administration.

In some embodiments, the present disclosure provides a method of treating endometrial cancer, e.g., hormone receptor positive endometrial cancer, estrogen receptor positive endometrial cancer, progesterone receptor positive endometrial cancer, estrogen receptor positive, progesterone receptor positive endometrial cancer in a subject in need thereof, the method comprising
(i) administering to the subject a therapeutically-effective amount of a first compound, wherein the first compound is a compound of formula (I):

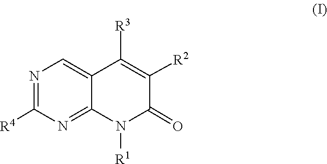

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen;
provided that, when R⁴ is —NR⁵R⁶ and one of R⁵ and R⁶ is pyridyl, then R² is not —C(O)CH₃,
or a pharmaceutically-acceptable salt thereof; and
   (ii) administering to the subject a therapeutically-effective amount of a second compound.

In some embodiments, the present disclosure provides a method of treating endometrial cancer, e.g., hormone receptor positive endometrial cancer, estrogen receptor positive endometrial cancer, progesterone receptor positive endometrial cancer, estrogen receptor positive, progesterone receptor positive endometrial cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7 oxo-7,8-dihydropyrido[2,3-d]pyrimidine-6-carbonitrile monolactate, wherein the subject received a therapy other than the compound for the endometrial cancer, e.g., hormone receptor positive endometrial cancer, estrogen receptor positive endometrial cancer, progesterone receptor positive endometrial cancer, estrogen receptor positive, progesterone receptor positive endometrial cancer prior to the administering, wherein the therapy was received after the subject was diagnosed with endometrial cancer, e.g., hormone receptor positive endometrial cancer, estrogen receptor positive endometrial cancer, progesterone receptor positive endometrial cancer, estrogen receptor positive, progesterone receptor positive endometrial cancer, and wherein the subject has not responded to the therapy prior to the administering; and wherein the administering comprises 3 weeks of once-daily administration.

In some embodiments, the present disclosure provides a method of treating endometrial cancer, e.g., hormone receptor positive endometrial cancer, estrogen receptor positive endometrial cancer, progesterone receptor positive endometrial cancer, estrogen receptor positive, progesterone receptor positive endometrial cancer in a subject in need thereof, the method comprising (i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the endometrial cancer, e.g., hormone receptor positive endometrial cancer, estrogen receptor positive endometrial cancer, progesterone receptor positive endometrial cancer, estrogen receptor positive, progesterone receptor positive endometrial cancer prior to the administering, wherein the therapy was received after the subject was diagnosed with endometrial cancer, e.g., hormone receptor positive endometrial cancer, estrogen receptor positive endometrial cancer, progesterone receptor positive endometrial cancer, estrogen receptor positive, progesterone receptor positive endometrial cancer, and wherein the subject has not responded to the therapy prior to the administering; and wherein the administering comprises 3 weeks of once-daily administration; and (ii) orally administering to the subject a therapeutically-effective amount of letrozole.

In some embodiments, the present disclosure provides a method of endometrial cancer, e.g., hormone receptor positive endometrial cancer, estrogen receptor positive endometrial cancer, progesterone receptor positive endometrial cancer, estrogen receptor positive, progesterone receptor positive endometrial cancer in a subject in need thereof, the method comprising (i) orally administering to the subject a solid pharmaceutical composition, wherein the solid pharmaceutical composition comprises 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate, wherein the subject received a therapy other than the compound for the endometrial cancer, e.g., hormone receptor positive endometrial cancer, estrogen receptor positive endometrial cancer, progesterone receptor positive endometrial cancer, estrogen receptor positive, progesterone receptor positive endometrial cancer prior to the administering, wherein the therapy was received after the subject was diagnosed with endometrial cancer, e.g., hormone receptor positive endometrial cancer, estrogen receptor positive endometrial cancer, progesterone receptor positive endometrial cancer, estrogen receptor positive, progesterone receptor positive endometrial cancer, and wherein the subject has not responded to the therapy prior to the administering, and wherein the administering comprises 3 weeks of once-daily administration; and (ii) orally administering to the subject a therapeutically-effective amount of megestrol acetate.

### BRIEF DESCRIPTION OF THE FIGURES

FIG. 1 shows KinMap for CDK 4/6 inhibitors: Compound 1, Comparator 1, Comparator 2, and Comparator 3.

FIG. 2 demonstrates Compound 1 treated cancer cell lines showed reduced Neutropenia in comparison to Comparator 1 treated cancer cell lines.

### DETAILED DESCRIPTION OF THE DISCLOSURE

Provided herein are compositions and methods for treating endometrial cancer by administering to a subject in need thereof a pharmaceutical composition, the pharmaceutical composition comprising in a unit dosage form a therapeutically-effective amount of a compound described herein or a pharmaceutically-acceptable salt thereof. In some embodiments, the methods further comprise administering a second pharmaceutical composition comprising in a unit dosage form a therapeutically-effective amount of a second compound, for example an estrogen blocking drug.

### Compounds of the Disclosure

A compound disclosed herein can be of the formula:

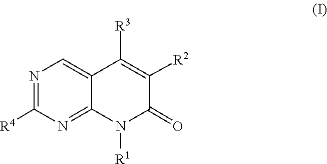

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS (O);R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
   provided that, when R⁴ is —NR⁵R⁶ and one of R⁵ and R⁶ is pyridyl, then R² is not —C(O)CH₃,
or a pharmaceutically-acceptable salt thereof.

A compound disclosed herein can be of the formula:

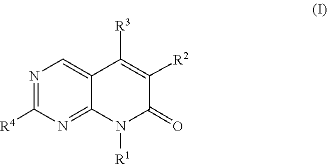

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is C₂-C₆ alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
or a pharmaceutically-acceptable salt thereof.

A compound disclosed herein can be a pharmaceutically-acceptable salt of the formula:

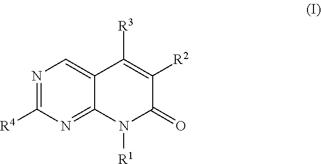

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen.

A compound disclosed herein can be lactate salt of the formula:

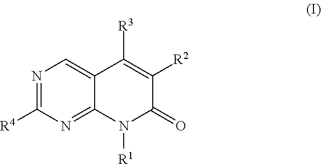

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS (O);R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen.

In some embodiments, R¹ is cycloalkyl. In some embodiments, R¹ is C₃-C₈ cycloalkyl. In some embodiments, R¹ is an unsubstituted cyclopentyl. In some embodiments, R¹ is a substituted cyclopentyl. In some embodiments, R² is CN. In some embodiments, R³ is hydrogen.

In some embodiments, R⁴ is —NR⁵R⁶. In some embodiments, one of R⁵ and R⁶ is hydrogen. In some embodiments, one of R⁵ and R⁶ is phenyl In some embodiments, one of R⁵ and R⁶ is phenyl substituted with heterocyclyl. In some embodiments, one of R⁵ and R⁶ is phenyl substituted with heterocyclyl, wherein the heterocyclyl contains at least one ring nitrogen atom. In some embodiments, one of R⁵ and R⁶ is phenyl substituted with C₃-C₈ heterocyclyl. In some embodiments, one of R⁵ and R⁶ is phenyl substituted with C₆ heterocyclyl. In some embodiments, one of R⁵ and R⁶ is phenyl substituted with piperazinyl, wherein the piperazinyl is unsubstituted or substituted. In some embodiments, one of R⁵ and R⁶ is phenyl substituted with piperazinyl, wherein the piperazinyl is substituted with an alkyl. In some embodiments, one of R⁵ and R⁶ is phenyl substituted with 4-methyl piperazinyl.

In some embodiments, R⁴ is

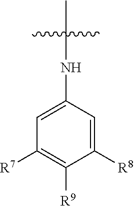

wherein:
R⁷ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen;
R⁸ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen; and
R⁹ is alkyl, alkenyl, alkynyl, cycloalkyl, aryl, heterocyclyl, heteroaryl, —OR⁵, —SR⁵, or —NR⁵R⁶, each of which is unsubstituted or substituted, or hydrogen.

In some embodiments, R⁷ is hydrogen. In some embodiments, R⁸ is hydrogen. In some embodiments, R⁹ unsubstituted or substituted heterocyclyl. In some embodiments, R⁹ is unsubstituted or substituted piperazinyl. In some embodiments, R⁹ is piperazinyl substituted with alkyl. In some embodiments, R⁹ is 4-methyl piperazinyl.

In some embodiments, the compound is a compound of formula (II)

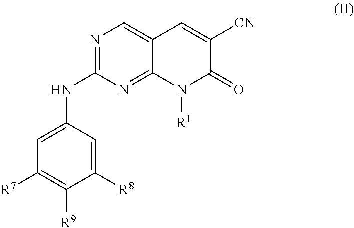

In some embodiments, the compound is a compound of formula (III)

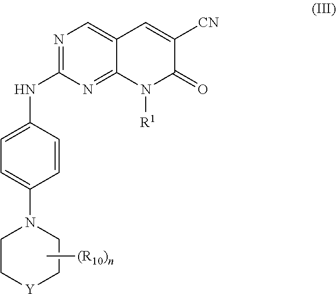

wherein:
Y is O, S, or NR¹¹;
each R¹⁰ is independently alkyl, alkenyl, alkynyl, cycloalkyl, aryl, heterocyclyl, heteroaryl, —OR⁵, —SR⁵, or —NR⁵R⁶, each of which is unsubstituted or substituted;
R¹¹ is alkyl, alkenyl, alkynyl, cycloalkyl, aryl, heterocyclyl, or heteroaryl, each of which is independently substituted or unsubstituted, or hydrogen or halogen; and
n is 0, 1, 2, 3, 4, 5, 6, 7, or 8.

In some embodiments, R¹ is cycloalkyl. In some embodiments, R¹ is C₃-C₈ cycloalkyl. In some embodiments, R¹ is an unsubstituted cyclopentyl. In some embodiments, R¹ is a substituted cyclopentyl.

In some embodiments, Y is NR¹¹. In some embodiments, R¹¹ is alkyl. In some embodiments, R¹¹ is methyl. In some embodiments, n is 0.

In some embodiments, the compound is of the formula:

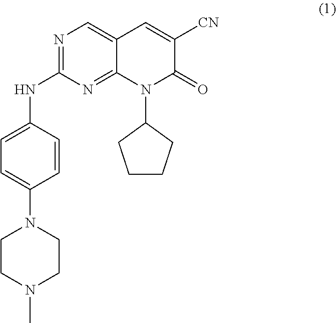 [SMILES: CN1CCN(c2ccc(Nc3ncc4cc(C#N)c(=O)n(C5CCCC5)c4n3)cc2)CC1]

8-cyclopentyl-2-((4-(4-methylpiperazin-1-yl)phenyl)amino)-7-oxo-7,8-dihydropyrido[2,3-d]pyrimidine-6-carbonitrile (Compound 1), or a pharmaceutically-acceptable salt thereof.

In some embodiments, the compound is in the form of a salt formed by combining a compound with lactic acid. In some embodiments, a compound disclosed herein is 8-cyclopentyl-2-((4-(4-methylpiperazin-1-yl)phenyl)amino)-7-oxo-7,8-dihydropyrido[2,3-d]pyrimidine-6-carbonitrile monolactate.

Several moieties described herein may be substituted or unsubstituted. Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

Non-limiting examples of alkyl groups include straight, branched, and cyclic alkyl groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxy propyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl or alkynylene groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl. Non-limiting examples of substituted aryl groups include 3,4-dimethylphenyl, 4-tert-butylphenyl, 4-cyclopropylphenyl, 4-diethylaminophenyl, 4-(trifluoromethyl)phenyl, 4-(difluoromethoxy)-phenyl, 4-(trifluoromethoxy)phenyl, 3-chlorophenyl, 4-chlorophenyl, 3,4-dichlorophenyl, 2-fluorophenyl, 2-chlorophenyl, 2-iodophenyl, 3-iodophenyl, 4-iodophenyl, 2-methylphenyl, 3-fluorophenyl, 3-methylphenyl, 3-methoxyphenyl, 4-fluorophenyl, 4-methylphenyl, 4-methoxyphenyl, 2,3-difluorophenyl, 3,4-difluorophenyl, 3,5-difluorophenyl, 2,3-dichlorophenyl, 3,4-dichlorophenyl, 3,5-dichlorophenyl, 2-hydroxyphenyl, 3-hydroxyphenyl, 4-hydroxyphenyl, 2-methoxyphenyl, 3-methoxyphenyl, 4-methoxyphenyl, 2,3-dimethoxyphenyl, 3,4-dimethoxyphenyl, 3,5-dimethoxyphenyl, 2,4-difluorophenyl, 2,5-difluorophenyl, 2,6-difluorophenyl, 2,3,4-trifluorophenyl, 2,3,5-trifluorophenyl, 2,3,6-trifluorophenyl, 2,4,5-trifluorophenyl, 2,4,6-trifluorophenyl, 2,4-dichlorophenyl dichlorophenyl, 2,5-dichlorophenyl, 2,6-dichlorophenyl, 3,4-dichlorophenyl, 2,3,4-trichlorophenyl, 2,3,5-trichlorophenyl, 2,3,6-trichlorophenyl, 2,4,5-trichlorophenyl, 3,4,5-trichlorophenyl, 2,4,6-trichlorophenyl, 2,3-dimethylphenyl, 2,4-dimethylphenyl, 2,5-dimethylphenyl, 2,6-dimethylphenyl, 2,3,4-trimethylphenyl, 2,3,5-trimethylphenyl, 2,3,6-trimethylphenyl, 2,4,5-trimethylphenyl, 2,4,6-trimethylphenyl, 2-ethylphenyl, 3-ethylphenyl, 4-ethylphenyl, 2,3-diethylphenyl, 2,4-diethylphenyl, 2,5-diethylphenyl, 2,6-diethylphenyl, 3,4-diethylphenyl, 2,3,4-triethylphenyl, 2,3,5-triethylphenyl, 2,3,6-triethylphenyl, 2,4,5-triethylphenyl, 2,4,6-triethylphenyl, 2-isopropylphenyl, 3-isopropylphenyl, and 4-isopropylphenyl.

Non-limiting examples of substituted aryl groups include 2-aminophenyl, 2-(N-methylamino)phenyl, 2-(N,N-dimethylamino)phenyl, 2-(N-ethylamino)phenyl, 2-(N,N-diethylamino)phenyl, 3-aminophenyl, 3-(N-methylamino)phenyl, 3-(N,N-dimethylamino)phenyl, 3-(N-ethylamino)phenyl, 3-(N,N-diethylamino)phenyl, 4-aminophenyl, 4-(N-methylamino)phenyl, 4-(N,N-dimethylamino)phenyl, 4-(N-ethylamino)phenyl, and 4-(N,N-diethylamino)phenyl.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

Non-limiting examples of heterocycles (heterocyclyl) include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]pyiazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl,

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutically Acceptable Salts

The method disclosed herein provides the use of pharmaceutically-acceptable salts of any compound described herein. Pharmaceutically-acceptable salts include, for example, acid-addition salts and base-addition salts. The acid that is added to the compound to form an acid-addition addition salt can be an organic acid or an inorganic acid. A base that is added to the compound to form a base-addition salt can be an organic base or an inorganic base.

Acid addition salts can arise from the addition of an acid to a compound disclosed herein. In some embodiments, the acid is organic. In some embodiments, the acid is inorganic. In some embodiments, the acid is lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, maleic acid, hydrochloric acid, hydrobromic acid, hydroiodic acid, nitric acid, nitrous acid, sulfuric acid, sulfurous acid, a phosphoric acid, or isonicotinic acid. In some embodiments, the salt is an acid addition salt with lactic acid. In some embodiments, the salt is an acid addition salt of 8-cyclopentyl-2-((4-(4-methylpiperazin-1-yl)phenyl)amino)-7-oxo-7,8-dihydropyrido[2,3-d]pyrimidine-6-carbonitrile with lactic acid.

In some embodiments, the salt is a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, a maleate salt, hydrochloride salt, a hydrobromide salt, a hydroiodide salt, a nitrate salt, a nitrite salt, a sulfate salt, a sulfite salt, a phosphate salt, isonicotinate salt. In some embodiments, the salt is a lactate salt. In some embodiments, the salt is a monolactate salt. In some embodiments, the salt is 8-cyclopentyl-2-((4-(4-methylpiperazin-1-yl)phenyl)amino)-7-oxo-7,8-dihydropyrido[2,3-d]pyrimidine-6-carbonitrile monolactate.

Metal salts can arise from the addition of an inorganic base to a compound disclosed herein. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

### Therapeutic Methods

Phosphoinositide 3-kinase (PI3 kinase or PI3K), protein kinase B (AKT), and mitogen-activated protein kinase (MAPK) pathways are central to many signaling pathways and are often de-regulated in cancers, including endometrial cancer. In some embodiments, a compound described herein can down-modulate these kinase pathways, or a portion thereof, for example, cyclin-dependent kinases (CDK). In some embodiments, overexpression of CDK, e.g., CDK 4/6 causes cell-cycle deregulation in cancers. In some embodiments, modulation of kinase pathways can result in the obstruction of proliferation signal receipt in cells, thus arresting tumor growth.

In some embodiments, a compound described herein can be an inhibitor of the cyclin-dependent kinase proteins CDK4. In some embodiments, a compound described herein can be an inhibitor of CDK6. In some embodiments, a compound described herein can be an inhibitor of CDK1. In some embodiments, a compound described herein can be an inhibitor of CDK2. In some embodiments, a compound described herein can be an inhibitor of CDK7. In some embodiments, a compound described herein can be an inhibitor of CDK9. In some embodiments, a compound described herein can be an inhibitor of CDK16. In some embodiments, a compound described herein can be an inhibitor of CDK17. In some embodiments, a compound described herein can be an inhibitor of CDK 4/6. In some embodiments, a compound described herein can be used for treating a cancer where other CDK4/6 inhibitors have failed. In some embodiments, a compound described herein can be an inhibitor of colony stimulating factor receptor 1 (CSF1R). In some embodiments, a compound described herein can be an inhibitor of proto-oncogene receptor tyrosine kinase KIT. In some embodiments, a compound described herein is an inhibitor of the AMPK-related protein kinase 5 (ARK5 or NUAK1) protein. ARK5 regulates Atk-dependent cell survival and migration (e.g., formation of metastases) through inhibition of cellular metabolism. ARK5 overexpression is found in multiple tumors and is associated with poor prognosis in metastatic breast cancer, multiple myeloma, and hepatocellular carcinoma. In some embodiments, inhibition of ARK5 induces cell death through PI3K/AKT/mTOR pathway. In some embodiments, a combination of CDK and ARK5 inhibitors has a synergistic effect on cancer cells by simultaneously inhibiting cell cycle (cytostatic) and cellular metabolism (cytotoxic) through CDK and ARK5, respectively.

In some embodiments, the inclusion of ARK5 in the functional activity profile of a compound described herein overcomes the emergence of resistance to CDK4/6 inhibitors due to the loss of retinoblastoma function and C-Myc overexpression. C-Myc expression is dependent on ARK5 activity and is involved in the pathogenesis of certain tumors. In some embodiments, the dual inhibitory effect of a compound described herein provides a therapeutic strategy to optimize efficacy of CDK 4/6 inhibition and reduce emergence of resistance. The present disclosure provides a method for the use of a compound disclosed herein, for example, for treating cancer.

In some embodiments, a compound disclosed herein, e.g., 8-cyclopentyl-2-((4-(4-methylpiperazin-1-yl)phenyl)amino)-7-oxo-7,8-dihydropyrido[2,3-d]pyrimidine-6-carbonitrile (Compound 1) or a pharmaceutically-acceptable salt thereof, may be active in numerous tumor types with acceptable and differentiated safety profile. FIG. 2 shows, in preclinical models, Compound 1 caused less neutropenia in comparison to an FDA approved CDK 4/6 inhibitor (Comparator 1 in TABLE 1). Compound 1 also had comparable IC₅₀ values for CDK4 and CDK6 as FDA approved CDK 4/6 inhibitor (Comparator 1, Comparator 2, and Comparator 3 in TABLE 1). Compound 1 had significantly lower IC₅₀ values (1 to 3 orders of magnitude lower) for CSF1R, ARK 5, KIT, than Comparator 1, Comparator 2, and Comparator 3. Compound (1) had significantly lower IC₅₀ values (1 to 3 orders of magnitude lower) for CDK 1, CDK 2, CDK 7, CDK 9, CDK 16, and CDK 17, than Comparator 1 and Comparator 2.

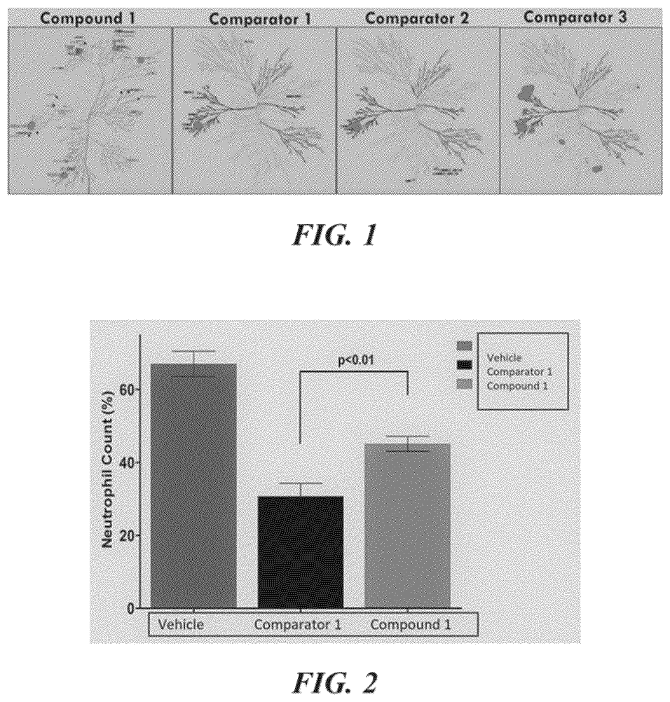

In some embodiments a compound disclosed herein, e.g., Compound 1 shows a wider inhibition profile compared to a kinase inhibitor that is not compound 1; e.g., as determined by KinMap (FIG. 1).

A method disclosed herein can be used to treat, for example, an infectious disease, a proliferative disease, a cancer, a solid tumor, or a liquid tumor. Non-limiting examples of tumors that are treatable by a combination of a compound described herein can include solid tumors, solid tumors that are refractory to prior treatment with conventional chemotherapy, and solid tumors that respond to initial chemotherapy but subsequently relapsed. In some embodiments, the tumor is an endometrial cancer tumor.

A tumor response due to a method disclosed herein can be measured based on the Response Evaluation Criteria in Solid Tumors (RECIST) classification of responses. To use RECIST, there must be at least one tumor that can be measured on x-rays, CT scans, or MRI scans. RECIST assigns four categories of response: complete response (CR), a partial response (PR), progressive disease (PD), and stable disease (SD). Key features of the RECIST include definitions of minimum size of measurable lesions, instructions on how many lesions to follow, and the use of unidimensional, rather than bidimensional, measures for overall evaluation of tumor burden.

In some embodiments, the present disclosure provides a method of treating endometrial cancer in a subject in need thereof, the method comprising administering to the subject a therapeutically-effective amount of a compound of formula (I), or a pharmaceutically-acceptable salt thereof. In some embodiments, the present disclosure provides a method of treating endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the endometrial cancer is a hormone receptor positive endometrial cancer. In some embodiments, the present disclosure provides a method of treating hormone receptor positive endometrial cancer in a subject in need thereof, the method comprising administering to the subject a therapeutically-effective amount of a compound of formula (I), or a pharmaceutically-acceptable salt thereof. In some embodiments, the present disclosure provides a method of treating hormone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating hormone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the endometrial cancer is a progesterone receptor positive endometrial cancer. In some embodiments, the present disclosure provides a method of treating progesterone receptor positive endometrial cancer in a subject in need thereof, the method comprising administering to the subject a therapeutically-effective amount of a compound of formula (I), or a pharmaceutically-acceptable salt thereof. In some embodiments, the present disclosure provides a method of treating progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the endometrial cancer is an estrogen receptor positive endometrial cancer. In some embodiments, the present disclosure provides a method of treating estrogen receptor positive endometrial cancer in a subject in need thereof, the method comprising administering to the subject a therapeutically-effective amount of a compound of formula (I), or a pharmaceutically-acceptable salt thereof. In some embodiments, the present disclosure provides a method of treating estrogen receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating estrogen receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the endometrial cancer is an estrogen receptor positive, progesterone receptor positive (ER+/PR+) endometrial cancer. In some embodiments, the present disclosure provides a method of treating ER+/PR+ endometrial cancer in a subject in need thereof, the method comprising administering to the subject a therapeutically-effective amount of a compound of formula (I), or a pharmaceutically-acceptable salt thereof. In some embodiments, the present disclosure provides a method of treating ER+/PR+ endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating ER+/PR+ endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiments, the endometrial cancer is a progesterone receptor positive (PR+) endometrial cancer. In some embodiments, the present disclosure provides a method of treating PR+ endometrial cancer in a subject in need thereof, the method comprising administering to the subject a therapeutically-effective amount of a compound of formula (I), or a pharmaceutically-acceptable salt thereof. In some embodiments, the present disclosure provides a method of treating PR+ endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof. In some embodiments, the present disclosure provides a method of treating PR+ endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate.

In some embodiment, the administering comprises an additional therapy, e.g., a second line, a third line, a fourth line, a fifth line, a sixth line, or a seventh line of therapy. In some embodiments, the subject received a therapy other than the compound for the endometrial cancer (e.g., the hormone receptor positive endometrial cancer) prior to the administering. In some embodiments, the therapy was received after the subject was diagnosed with the endometrial cancer. In some embodiments, the therapy was received after the subject was diagnosed with the hormone receptor positive endometrial cancer. In some embodiments, the therapy was received after the subject was diagnosed with the estrogen receptor positive endometrial cancer. In some embodiments, the subject did not respond to the therapy. In some embodiments, the subject experienced a relapse of the endometrial cancer after the therapy. In some embodiments, the subject experienced a relapse of the hormone receptor positive endometrial cancer after the therapy. In some embodiments, the subject experienced a relapse of the estrogen receptor positive endometrial cancer after the therapy.

### Combination Therapy

The present disclosure also provides a combination and method for using such of a compound disclosed herein, for example, 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, with an estrogen blocker and/or an aromatase inhibitor, for example letrozole.

In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a second compound, e.g., a drug. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor modulator. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an estrogen receptor blocker, e.g., an aromatase inhibitor such as letrozole. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a progestin such as megestrol or esters thereof (e.g., megestrol acetate). In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with estrogen receptor degrader. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with an angiogenesis inhibitor. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a vascular endothelial growth factor (VEGF) inhibitor. In some embodiments, a compound of the disclosure, e.g., a compound of formula (I), such as 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, is administered in combination with a phosphoinositide 3-kinase (PI3K) inhibitor.

### Estrogen Receptor Blockers

Estrogens stimulate or maintain the growth of some cancers (e.g., endometrial cancer). Treatment of endometrial cancer thought to be hormonally responsive (e.g., estrogen and/or progesterone receptor positive) is aimed at decreasing estrogen levels or inhibiting estrogen effects. In some embodiments, these interventions lead to decreased tumor mass or delayed progression of tumor growth.

Estrogen blockers block the production of estrogens (e.g., estradiol) or prevent estrogens (e.g. estradiol) from mediating biological effects in the body. Estrogen blockers act by blocking the estrogen receptor and/or directly inhibiting or suppressing estrogen production. Estrogen blockers are divided into classes by whether the estrogen blockers reduce the production of estrogen (aromatase inhibitors and antigonadotropins) or whether the estrogen blockers reduce the response to estrogen (antiestrogens and estrogen antagonists). By producing less estrogen, or by blocking the response to estrogen, estrogen blockers slow or inhibit the growth of cancer cells that require estrogen to stimulate growth (e.g., endometrial cells, uterine cells). Non-limiting examples of estrogen blockers include aromatase inhibitors, e.g., letrozole (Femara), anastrozole (Arimidex), exemestane (Aromasin), tamoxifen, testolactone (Teslac), ethamoxytriphetol, clomifene, and raloxifene.

Non-limiting examples of cells whose activity can be modulated by a combination therapy of a compound described herein and an estrogen blocker include secretory cells, cells with cilia, basal cells, red blood cells, mesenchymal cells, pluripotential mesenchymal cells, predecidual cells, epithelial cells, histiocytes, granulocytes, glandular cells, stromal cells, normal endometrial cells (NEMCs), atypical endometrial cells (AEMCs), and endometrial carcinoma cells (EMCCs).

Non-limiting examples of tumors that are treatable by a combination of a compound described herein and an estrogen blocker include solid tumors, solid tumors that are refractory to prior treatment with conventional chemotherapy, and solid tumors that respond to initial chemotherapy and subsequently relapse, as evidenced, e.g., by disease progression.

Non-limiting examples of cancers that are treatable by a combination of a compound described herein and an estrogen blocker include endometrial cancer, endometrial cancer that is hormone receptor positive (HR+), endometrial cancer that is estrogen-receptor positive (ER+), estrogen receptor positive, progesterone receptor positive (ER+/PR+), endometrial cancer that is progesterone receptor positive (PR+), endometrial cancer that is refractory to prior treatment with conventional chemotherapy, and endometrial cancer that responds to initial chemotherapy but subsequently relapses.

### Aromatase Inhibitors

In postmenopausal women, estrogens are mainly derived from the action of the aromatase enzyme. This enzyme converts adrenal androgens (e.g., androstenedione and testosterone) to estrone and estradiol. The suppression of estrogen biosynthesis in peripheral tissues and in the cancer tissue is achieved by inhibiting the aromatase enzyme.

Letrozole is a nonsteroidal competitive inhibitor of the aromatase enzyme system. Letrozole inhibits the conversion of androgens to estrogens. Letrozole selectively inhibits gonadal steroidogenesis but has no significant effect on adrenal mineralocorticoid or glucocorticoid synthesis. Letrozole inhibits the aromatase enzyme by competitively binding to the heme of the cytochrome P450 subunit of the enzyme, resulting in a reduction of estrogen biosynthesis. Treatment of women with letrozole significantly lowers serum estrone, estradiol and estrone sulfate.

The structure of letrozole is depicted below.

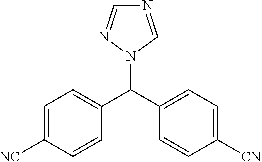 [SMILES: N#Cc1ccc(C(c2ccc(C#N)cc2)n2cncn2)cc1]

4,4′-((1H-1,2,4-triazol-1-yl)methylene)dibenzonitrile. Pharmaceutically-acceptable salts of 4,4′-((1H-1,2,4-triazol-1-yl)methylene)dibenzonitrile can also be used.

### Progestins

Progestins are synthetic forms of the naturally-occurring hormone progesterone. Progestins counteract estrogen effects on the body and function similarly to estrogen blockers. Non-limiting examples of progestins include megestrol acetate, acetomepregenol, chlormadinone acetate, cyproterone acetate, danazol, drospirenone, gestrinone, levonorgestrel, medrogestone, norethisterone, norethisterone acetate, norgestrel, oxendolone, osaterone acetate, trimegestone. norethindrone, norethindrone acetate, norethynodrel, ethynodiol diacetate, a third desogestrel, gestodene, norgestimate, dienogest, nestorone, and nomegestrol acetate.

Megestrol acetate is a synthetic derivative of progesterone. Megestrol binds to progesterone receptors and changes the hormone balance in the body, which may inhibit or stop cancers associated with estrogen.

The structure of megestrol acetate is depicted below.

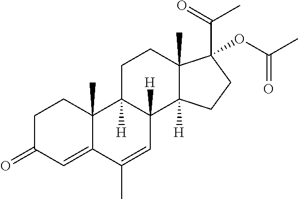

17-Hydroxy-6-methyl pregna-4,6-diene-3,20-dione acetate.

### Selective Estrogen Receptors Degraders

A selective estrogen receptor degrader or down-regulator (SERD) can bind to the estrogen receptor and cause the estrogen receptor to be degraded and thus downregulated. SERDs are used to treat estrogen receptor-sensitive or progesterone receptor-sensitive cancers along with other similar classes of drugs such as estrogen blockers and aromatase inhibitors.

### Angiogenesis Inhibitors and Vascular Endothelial Growth Factor Inhibitors

Angiogenesis inhibitors interfere with blood vessel formation. Normally, the angiogenesis stimulating and inhibiting effects of these chemical signals are balanced so that blood vessels form only when and where vessels are needed. However, these signals can become unbalanced, causing increased blood vessel growth that can lead to abnormal conditions or disease, such as cancer.

Vascular endothelial growth factors (VEGF) are angiogenic factors that are growth factors for vascular endothelial cells. When VEGF and other endothelial growth factors bind to receptors on endothelial cells, signals within these cells are initiated that promote the growth and survival of new blood vessels.

Angiogenesis inhibitors interfere with various steps in blood vessel growth. Some angiogenesis inhibitors are monoclonal antibodies that specifically recognize and bind to VEGF to block the binding of VEGF to the VEGF receptor. Other angiogenesis inhibitors bind to VEGF, the VEGFs receptor, other receptors on the surface of endothelial cells, or to other proteins in the downstream signaling pathways. Some angiogenesis inhibitors are immunomodulatory drugs—agents that stimulate or suppress the immune system—that also have antiangiogenic properties. Non-limiting examples of VEGF inhibitors include bevacizumab (Avastin), sorafenib (Nexavar), sunitinib (Sutent), nilotinib (Tasigna), pazopanib (Votrient), and dasatinib (Sprycel).

### PI3K Inhibitors

Phosphoinositide 3-kinase (PI3K) inhibitors inhibit one or more of the phosphoinositide 3-kinase enzymes. These enzymes form part of the PI3K/AKT/mTOR pathway, which is a pathway involved in cell growth and survival, and other processes that are frequently activated in many cancers. By inhibiting these enzymes, PI3K inhibitors cause cell death, inhibit the proliferation of malignant cells, and interfere with several signaling pathways. PI3K inhibitors are usually given to treat certain cancers that have relapsed or are unresponsive to other cancer treatments. Non-limiting examples of PI3K inhibitors include alpelisib, copanlisib, duvelisib, and idelalisib.

### Administration of Compounds of the Disclosure

### Compound 1

A compound disclosed herein, for example, 8-cyclopentyl-2-((4-(4-methylpiperazin-1-yl)phenyl)amino)-7-oxo-7,8-dihydropyrido[2,3-d]pyrimidine-6-carbonitrile (Compound 1), can be formulated as a capsule. A capsule may be a hard capsule. A capsule may be a soft capsule. A capsule may be a soft gelatin capsule. In some embodiments, a compound disclosed herein can be formulated as a hard capsule, the hard capsule comprising an amount of 8-cyclopentyl-2-((4-(4-methylpiperazin-1-yl)phenyl)amino)-7-oxo-7,8-dihydropyrido[2,3-d]pyrimidine-6-carbonitrile monolactate equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Alternatively, a compound described herein can be formulated as a tablet.

In some embodiments, the present disclosure provides a pharmaceutical composition comprising, in a unit dosage form, an amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, equivalent to 40 mg of a compound described herein.

In some embodiments, the present disclosure provides a pharmaceutical composition comprising, in a unit dosage form, an amount of 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof, equivalent to 60 mg of a compound described herein.

In some embodiments, the present disclosure provides a pharmaceutical composition comprising, in a unit dosage form, an amount of 8-cyclopentyl-2-((4-(4-methylpiperazin-1-yl)phenyl)amino)-7-oxo-7,8-dihydropyrido[2,3-d]pyrimidine-6-carbonitrile monolactate, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile.

In some embodiments, the present disclosure provides a pharmaceutical composition comprising, in a unit dosage form, 48.4 mg of 8-cyclopentyl-2-((4-(4-methylpiperazin-1-yl)phenyl)amino)-7-oxo-7,8-dihydropyrido[2,3-d]pyrimidine-6-carbonitrile monolactate.

In some embodiments, 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt is administered in oral capsules, swallowed with water in the morning in a fasted state, at least 1 hour before ingesting food. In some embodiments, a morning dose is taken after an overnight fast an hour before ingesting food. In some embodiments, a compound described herein is administered every day. In some embodiments, a compound described herein is administered every day for 4 weeks. In some embodiments, a compound described herein is on a 4-week cycle of: (i) a continuous, three-week period of once-daily administration; and (ii) immediately following the three-week period, one week of no administration.

### Letrozole

A compound disclosed herein, for example, letrozole, can be formulated as tablet. In some embodiments, letrozole can be formulated as a tablet, the tablet comprising, in a unit dosage form, a therapeutically-effective amount of letrozole, and a pharmaceutically-acceptable excipient.

In some embodiments, letrozole is provided as 2.5 mg tablets for oral administration. The tablets can be colored, e.g., yellow, and can be uncoated or film-coated. Inactive ingredients may include colloidal silicon dioxide, ferric oxide, hydroxypropyl methylcellulose, lactose monohydrate, magnesium stearate, maize starch, microcrystalline cellulose, polyethylene glycol, sodium starch glycolate, talc, and titanium dioxide. In some embodiments, letrozole is given at a dose of one 2.5 mg tablet administered once a day without regards to food.

### Megestrol Acetate

A compound disclosed herein, for example, megestrol acetate, can be formulated as a suspension, for example a suspension suitable for oral administration. In some embodiments, megestrol acetate oral suspension contains 625 mg of megestrol acetate per 5 mL (125 mg/mL). In some embodiments, megestrol acetate oral suspension contains 800 mg per 20 mL (40 mg/mL). In some embodiments, megestrol acetate oral suspension contains one or more of the following inactive ingredients: alcohol (up to 0.06% v/v), lime flavor, citric acid monohydrate, decusate sodium, hydroxypropyl methylcellulose, natural and artificial lemon flavor, purified water, sodium benzoate, sodium citrate, dihydrate, and sucrose.

In some embodiments, megestrol acetate can be formulated as a tablet. In some embodiments, tablets contain 20 mg megestrol acetate. In some embodiments, tablets contain 40 mg megestrol acetate. In some embodiments, megestrol acetate tablets contain one or more of the following inactive ingredients: acacia spray dried, colloidal silicon dioxide, corn starch, di-calcium phosphate dihydrate powder, lactose hydrous impalpable, magnesium stearate and pregelatinized starch.

### Pharmaceutical Compositions

A pharmaceutical composition of the disclosure can be a combination of any pharmaceutical compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, inhalation, oral, parenteral, ophthalmic, otic, subcutaneous, transdermal, nasal, intravitreal, intratracheal, intrapulmonary, transmucosal, vaginal, and topical administration.

Formulations can be modified depending upon the route of administration chosen. Pharmaceutical compositions comprising a compound described herein can be manufactured, for example, by mixing, dissolving, emulsifying, encapsulating, entrapping, or compression processes.

Non-limiting examples of dosage forms suitable for use in a method disclosed herein include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, and any combination thereof.

Pharmaceutical compositions can be formulated by combining the active compounds with pharmaceutically-acceptable carriers or excipients. Non-limiting examples of pharmaceutically-acceptable excipients suitable for use in the method disclosed herein include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, anti-microbial agents, plant cellulosic material and spheronization agents, and any combination thereof.

Non-limiting examples of pharmaceutically-acceptable carriers include saline solution, Ringer's solution and dextrose solution. Further carriers include sustained release preparations such as semipermeable matrices of solid hydrophobic polymers containing the compound disclosed herein, where the matrices are in the form of shaped articles, such as films, liposomes, microparticles, and microcapsules.

For oral administration, pharmaceutical compositions can be formulated by combining the active compounds with pharmaceutically-acceptable carriers or excipients into a unit dosage form which can be solid or liquid. Non-limiting examples of oral solid forms include tablets, powders, pills, dragees, capsules, liquids, gels, syrups, elixirs, slurries, or suspensions for oral ingestion by a subject. Pharmaceutical preparations for oral use can be obtained by mixing one or more solid excipients with one or more compounds described herein, optionally grinding the resulting mixture, and processing the mixture of granules, after adding suitable auxiliaries, if desired, to obtain tablets or dragee cores. Cores can be provided with suitable coatings. For this purpose, concentrated sugar solutions can be used. The solutions can contain an excipient such as gum arabic, talc, polyvinylpyrrolidone, carbopol gel, polyethylene glycol, or titanium dioxide, lacquer solutions, and suitable organic solvents or solvent mixtures. Dyestuffs or pigments can be added to the tablets or dragee coatings, for example, for identification or to characterize different combinations of active compound doses.

Pharmaceutical preparations that can be used orally include push-fit capsules made of gelatin and soft, sealed capsules made of gelatin and a plasticizer, such as glycerol or sorbitol. Pharmaceutical preparations that can be used orally include coated and uncoated tablets. In some embodiments, the capsule comprises a hard gelatin capsule, the capsule comprising one or more of pharmaceutical, bovine, and plant gelatins. A gelatin can be alkaline-processed. The capsule or tablet can contain the active ingredients in admixture with filler such as lactose, binders such as starches, or lubricants such as talc or magnesium stearate, and stabilizers. In soft capsules, the active compounds can be dissolved or suspended in suitable liquids, such as fatty oils, liquid paraffin, or liquid polyethylene glycols. Stabilizers can be added. All formulations for oral administration are provided in dosages suitable for such administration.

For oral administration of a liquid unit dosage form, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino)propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Parenteral injections can be formulated for bolus injection or continuous infusion. The pharmaceutical compositions can be in a form suitable for parenteral injection as a sterile suspension, solution or emulsion in oily or aqueous vehicles such as saline or water for injection, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives. The compounds of the disclosure can be applied topically to the skin, or a body cavity, for example, oral, vaginal, bladder, cranial, spinal, thoracic, or pelvic cavity of a subject. The compounds of the disclosure can be applied to an accessible body cavity.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, and synthetic polymers such as polyvinylpyrrolidone and PEG. In suppository forms of the compositions, a low-melting wax such as a mixture of fatty acid glycerides, optionally in combination with cocoa butter, can be used.

For buccal or sublingual administration, the compositions can be tablets, lozenges, or gels.

Formulations suitable for transdermal administration of the active compounds can employ transdermal delivery devices and transdermal delivery patches, and can be lipophilic emulsions or buffered aqueous solutions, dissolved or dispersed in a polymer or an adhesive. Such patches can be constructed for continuous, pulsatile, or on demand delivery of pharmaceutical compounds. Transdermal delivery can be accomplished by iontophoretic patches. Transdermal patches can provide controlled delivery. The rate of absorption can be slowed by using rate-controlling membranes or by trapping the compound within a polymer matrix or gel. Absorption enhancers can be used to increase absorption. An absorption enhancer or carrier can include absorbable pharmaceutically-acceptable solvents to assist passage through the skin. For example, transdermal devices can be in the form of a bandage comprising a backing member, a reservoir containing compounds and carriers, a rate controlling barrier to deliver the compounds to the skin of the subject at a controlled and predetermined rate over a prolonged period of time, and adhesives to secure the device to the skin or the eye.

For administration by inhalation, the active compounds can be in a form as an aerosol, a vapor, a mist, or a powder. Inhalation can occur through by nasal delivery, oral delivery, or both.

Nasal or intranasal administration involves insufflation of compounds through the nose, for example, nasal drops and nasal sprays. This route of administration can result in local and/or systemic effects. Inhaler or insufflator devices can be used for nose-to-lung delivery of compounds described herein.

A pharmaceutical composition can be administered in a local or systemic manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

Non-limiting examples of pharmaceutically-acceptable excipients suitable for use in the disclosure include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, solubilizers, stabilizers, tonicity enhancing agents, buffers and any combination thereof.

A composition of the disclosure can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 h.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 h.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Subjects can be, for example, elderly adults, adults, adolescents, pre-adolescents, children, toddlers, infants, neonates, and non-human animals. In some embodiments, a subject is a patient.

A method disclosed herein relates to administering the compound disclosed herein as part of a pharmaceutical composition. In some embodiments, compositions of a compound disclosed herein can comprise a liquid comprising an active agent in solution, in suspension, or both. Liquid compositions can include gels. In some embodiments, the liquid composition is aqueous. Alternatively, the composition can be an ointment. In some embodiments, the composition is an in situ gellable aqueous composition. In some embodiments, the composition is an in situ gellable aqueous solution.

A pharmaceutically-acceptable excipient can be present in a pharmaceutical composition at a mass of between about 0.1% and about 99% by mass of the composition. For example, a pharmaceutically-acceptable excipient can be present in a pharmaceutical composition at a mass of between about 0.1% and about 95%, between about 0.1% and about 90%, between about 0.1% and about 85%, between about 0.1% and about 80%, between about 0.1% and about 75%, between about 0.1% and about 70%, between about 0.1% and about 65%, between about 0.1% and about 60%, between about 0.1% and about 55%, between about 0.1% and about 50%, between about 0.1% and about 45%, between about 0.1% and about 40%, between about 0.1% and about 35%, between about 0.1% and about 30%, between about 0.1% and about 25%, between about 0.1% and about 20%, between about 0.1% and about 15%, between about 0.1% and about 10%, between about 0.1% and about 5%, or between about 0.1% and about 1%, by mass of the formulation.

A pharmaceutically-acceptable excipient can be present at about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% by mass of the formulation.

### Dosing

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for parenteral injection can be presented in unit dosage form, for example, in ampoules, or in multi-dose containers with a preservative.

A compound described herein can be present in a composition in a range of from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, from about 30 mg to about 35 mg, from about 35 mg to about 40 mg, from about 40 mg to about 45 mg, from about 45 mg to about 50 mg, from about 50 mg to about 55 mg, from about 55 mg to about 60 mg, from about 60 mg to about 65 mg, from about 65 mg to about 70 mg, from about 70 mg to about 75 mg, from about 75 mg to about 80 mg, from about 80 mg to about 85 mg, from about 85 mg to about 90 mg, from about 90 mg to about 95 mg, from about 95 mg to about 100 mg, from about 100 mg to about 125 mg, from about 125 mg to about 150 mg, from about 150 mg to about 175 mg, from about 175 mg to about 200 mg, from about 200 mg to about 225 mg, from about 225 mg to about 250 mg, from about 250 mg to about 300 mg, from about 300 mg to about 325 mg, from about 325 mg to about 350 mg, from about 350 mg to about 375 mg, from about 375 mg to about 400 mg, from about 400 mg to about 425 mg, from about 425 mg to about 450 mg, from about 450 mg to about 475 mg, or from about 475 mg to about 500 mg.

A compound described herein can be present in a composition in an amount of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 225 mg, about 250 mg, 300 about 300 mg, about 325 mg, about 350 mg, about 375 mg, about 400 mg, about 425 mg, about 450 mg, about 475 mg, or about 500.

A compound described herein can be administered to a subject in an amount of about 0.1 mg/kg to about 500 mg/kg, about 1 mg/kg to about 500 mg/kg, about 0.1 mg/kg to about 300 mg/kg, about 1 mg/kg to about 300 mg/kg, or about 0.1 mg/kg to about 30 mg/kg. In some embodiments, the compound disclosed herein is administered to a subject in an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, about 100 mg/kg, about 120 mg/kg, about 150 mg/kg, about 160 mg/kg, about 180 mg/kg, about 200 mg/kg, about 240 mg/kg, about 250 mg/kg, about 300 mg/kg, about 350 mg/kg, about 360 mg/kg, about 400 mg/kg, about 450 mg/kg, about 500 mg/kg, or about 600 mg/kg of the subject.

### Dosing Regimens

A dosing regimen disclosed herein can be, for example, once a day, twice a day, thrice a day, once a week, twice a week, or thrice a week. In some embodiments, a compound disclosed herein is administered once daily. In some embodiments, a compound disclosed herein is administered once daily for 28 days (one cycle). In some embodiments, a compound disclosed herein is administered once daily in one or more 28 day cycles. In some embodiments, a compound disclosed herein is administered in a four-week cycle of consecutive once daily administration for three weeks, followed by one week with no administrations.

In some embodiments, the present disclosure provides a method of treating endometrial cancer in a subject in need thereof, the method comprising administering to the subject a therapeutically-effective amount of a compound of formula (I)

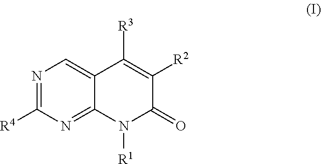

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
provided that, when R⁴ is —NR⁵R⁶ and one of R⁵ and R⁶ is pyridyl, then R² is not —C(O)CH₃,
or a pharmaceutically-acceptable salt thereof;
wherein the administering is once daily for at least 4 weeks.

In some embodiments, the present disclosure provides a method of treating endometrial cancer in a subject in need thereof, the method comprising administering to the subject a therapeutically-effective amount of a compound of formula (I)

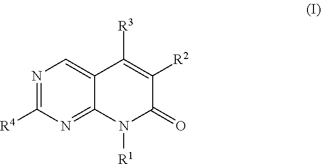

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
provided that, when R⁴ is —NR⁵R⁶ and one of R⁵ and R⁶ is pyridyl, then R² is not —C(O)CH₃,
or a pharmaceutically-acceptable salt thereof;
   wherein the administering is a 4-week cycle of: (i) a continuous, three-week period of once-daily administration; and ii) immediately following the three-week period, one week of no administration.

A compound described herein can be administered before, during, or after the occurrence of a disease or condition, and the timing of administering the composition containing a compound can vary. For example, a compound can be used as a prophylactic and can be administered continuously to subjects with a propensity to conditions or diseases to lessen or reduce a likelihood of the occurrence of the disease or condition. A compound and composition can be administered to a subject during or as soon as possible after the onset of the symptoms. The administration of a compound can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein.

A compound can be administered as soon as is practical after the onset of a disease or condition is detected or suspected, and for a length of time necessary for the treatment of the disease, such as, for example, from about 1 month to about 3 months. In some embodiments, the length of time a compound can be administered can be about 1 day, about 2 days, about 3 days, about 4 days, about 5 days, about 6 days, about 1 week, about 2 weeks, about 3 weeks, about 4 weeks, about 1 month, about 5 weeks, about 6 weeks, about 7 weeks, about 8 weeks, about 2 months, about 9 weeks, about 10 weeks, about 11 weeks, about 12 weeks, about 3 months, about 13 weeks, about 14 weeks, about 15 weeks, about 16 weeks, about 4 months, about 17 weeks, about 18 weeks, about 19 weeks, about 20 weeks, about 5 months, about 21 weeks, about 22 weeks, about 23 weeks, about 24 weeks, about 6 months, about 7 months, about 8 months, about 9 months, about 10 months, about 11 months, about 1 year, about 13 months, about 14 months, about 15 months, about 16 months, about 17 months, about 18 months, about 19 months, about 20 months, about 21 months, about 22 months about 23 months, about 2 years, about 2.5 years, about 3 years, about 3.5 years, about 4 years, about 4.5 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, or about 25 years. The length of treatment can vary for each subject.

A dosing schedule for administration of a compound described herein can be consistent for the length of the dosing regimen. For example, a compound can be administered daily. Alternatively, or in addition to, a dosing schedule for administration of a compound described herein can include portions of time where dosing is paused. For example, a compound can be administered every day for 3 weeks and then not be administered for one week.

A dosing schedule for administration of a compound described herein can include once daily (QD), twice daily (BID), three times daily (TID), four times daily (QID), once weekly, twice weekly, three times weekly, once monthly, twice monthly, and once every other month. For example, a daily dose can be given in a single dose or divided into multiple doses to be administered in intervals, e.g., twice daily, three times daily, and the like. For example, a daily dose of 100 mg can be given, for example, once daily (100 mg), twice daily (50 mg per dose).

Multiple therapeutic agents can be administered in any order or simultaneously. In some embodiments, a compound of the disclosure is administered in combination with, before, or after treatment with another therapeutic agent, e.g., a drug, such as an aromatase inhibitor. In some embodiments, a compound of the disclosure is administered at regular intervals, such as, for example, once daily, twice daily, thrice daily, etc. and the second therapeutic agent is administered daily or intermittently or on an as-needed basis. If simultaneously, the multiple therapeutic agents can be provided in a single, unified form, or in multiple forms, for example, as multiple separate unit dosage forms. The agents can be packed together or separately, in a single package or in a plurality of packages. One or all of the therapeutic agents can be given in multiple doses. If not simultaneous, the timing between the multiple doses can vary to as much as about a month.

### Compound 1 Dosing Regimens

A dosing regimen disclosed herein can be, for example, one dose of 40 mg, one dose of 60 mg, one dose of 80 mg, one dose of 120 mg; one dose of 160 mg, or one dose of 200 mg of oral 8-cyclopentyl-2-((4-(4-methylpiperazin-1-yl)phenyl)amino)-7-oxo-7,8-dihydropyrido[2,3-d]pyrimidine-6-carbonitrile per day. Alternatively, the dosing regimen disclosed herein can be, for example, 40 mg twice daily, 60 mg twice daily, 80 mg twice daily, or 100 mg twice daily. In some embodiments, the dosing is oral. In some embodiments, a dosing regimen can range from about 200 mg to about 500 mg per day, for example about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg per day

A dosing regimen disclosed herein can be, for example, one dose of 40 mg, two doses of 40 mg, three doses of 40 mg, four doses of 40 mg, five doses of 40 mg, six doses of 40 mg, seven doses of 40 mg, eight doses of 40 mg, 9 doses of 40 mg, 10 doses of 40 mg, 11 doses of 40 mg, or 12 doses of 40 mg of oral 8-cyclopentyl-2-((4-(4-methylpiperazin-1-yl)phenyl)amino)-7-oxo-7,8-dihydropyrido[2,3-d]pyrimidine-6-carbonitrile per day. In some embodiments, the dosing is oral.

A dosing regimen disclosed herein can be, for example, one dose of 60 mg, two doses of 60 mg, three doses of 60 mg, four doses of 60 mg, five doses of 60 mg, six doses of 60 mg, seven doses of 60 mg, eight doses of 60 mg of oral 8-cyclopentyl-2-((4-(4-methylpiperazin-1-yl)phenyl)amino)-7-oxo-7,8-dihydropyrido[2,3-d]pyrimidine-6-carbonitrile per day. In some embodiments, the dosing is oral.

In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily. In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily for 28 days (one cycle). In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered once daily in one or more 28 day cycles. In some embodiments, 8-cyclopentyl-2-((4-(4-methylpiperazin-1-yl)phenyl)amino)-7-oxo-7,8-dihydropyrido[2,3-d]pyrimidine-6-carbonitrile is administered in a four-week cycle of consecutive once daily administration for three weeks, followed by one week with no administrations.

In some embodiments, the present disclosure provides a method of treating hormone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is once daily for at least 4 weeks.

In some embodiments, the present disclosure provides a method of treating hormone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is a 4-week cycle of: (i) a continuous, three-week period of once-daily administration; and ii) immediately following the three-week period, one week of no administration.

In some embodiments, the present disclosure provides a method of treating hormone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is once daily for at least 4 weeks.

In some embodiments, the present disclosure provides a method of treating hormone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is a 4-week cycle of: (i) a continuous, three-week period of once-daily administration; and ii) immediately following the three-week period, one week of no administration.

In some embodiments, the present disclosure provides a method of treating progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is once daily for at least 4 weeks.

In some embodiments, the present disclosure provides a method of treating progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is a 4-week cycle of: (i) a continuous, three-week period of once-daily administration; and ii) immediately following the three-week period, one week of no administration.

In some embodiments, the present disclosure provides a method of treating progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is once daily for at least 4 weeks.

In some embodiments, the present disclosure provides a method of treating progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is a 4-week cycle of: (i) a continuous, three-week period of once-daily administration; and ii) immediately following the three-week period, one week of no administration.

In some embodiments, the present disclosure provides a method of treating estrogen receptor positive, progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is once daily for at least 4 weeks.

In some embodiments, the present disclosure provides a method of treating estrogen receptor positive, progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is a 4-week cycle of: (i) a continuous, three-week period of once-daily administration; and ii) immediately following the three-week period, one week of no administration.

In some embodiments, the present disclosure provides a method of treating estrogen receptor positive, progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is once daily for at least 4 weeks.

In some embodiments, the present disclosure provides a method of treating estrogen receptor positive, progesterone receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is a 4-week cycle of: (i) a continuous, three-week period of once-daily administration; and ii) immediately following the three-week period, one week of no administration.

In some embodiments, the present disclosure provides a method of treating estrogen receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is once daily for at least 4 weeks.

In some embodiments, the present disclosure provides a method of treating estrogen receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is a 4-week cycle of: (i) a continuous, three-week period of once-daily administration; and ii) immediately following the three-week period, one week of no administration.

In some embodiments, the present disclosure provides a method of treating estrogen receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is once daily for at least 4 weeks.

In some embodiments, the present disclosure provides a method of treating estrogen receptor positive endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is a 4-week cycle of: (i) a continuous, three-week period of once-daily administration; and ii) immediately following the three-week period, one week of no administration.

In some embodiments, the present disclosure provides a method of treating recurrent metastatic low-grade endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is once daily for at least 4 weeks.

In some embodiments, the present disclosure provides a method of treating recurrent metastatic low-grade endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile or a pharmaceutically-acceptable salt thereof, wherein the administering is a 4-week cycle of: (i) a continuous, three-week period of once-daily administration; and ii) immediately following the three-week period, one week of no administration.

In some embodiments, the present disclosure provides a method of treating recurrent metastatic low-grade endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is once daily for at least 4 weeks.

In some embodiments, the present disclosure provides a method of treating recurrent metastatic low-grade endometrial cancer in a subject in need thereof, the method comprising administering to the subject a therapeutically-effective amount of 8-cyclopentyl-2-((4-(4-methylpiperazin-1 yl)phenyl)amino)-7-oxo-7,8-dihydropyrido[2,3-d]pyrimidine-6-carbonitrile monolactate, wherein the administering is a 4-week cycle of: (i) a continuous, three-week period of once-daily administration; and ii) immediately following the three-week period, one week of no administration.

### Letrozole Dosing Regimens

A dosing regimen disclosed herein can be, for example, once a day, twice a day, thrice a day, once a week, twice a week, or thrice a week. In some embodiment, the dosing is oral. In some embodiments, a suitable amount letrozole can range from about 0.1 mg to about 100 mg per day, for example about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, or about 50 mg per day.

### Compound 1 and Letrozole Dosing Regimen

A dosing regimen disclosed herein can be, for example, a combination of 8-cyclopentyl-2-((4-(4-methylpiperazin-1 yl)phenyl)amino)-7-oxo-7,8-dihydropyrido[2,3-d]pyrimidine-6-carbonitrile and letrozole. In some embodiments, the dosing is oral. In some embodiments, a suitable amount of 8-cyclopentyl-2-((4-(4-methylpiperazin-1 yl)phenyl)amino)-7-oxo-7,8-dihydropyrido[2,3-d]pyrimidine-6-carbonitrile can range from about 40 mg to about 500 mg per day, for example about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg per day, and a suitable amount of letrozole can range from about 0.1 mg to 20 mg per day, for example about 0.1 mg, about 0.2 mg, about 0.3 mg, about 0.4 mg, about 0.5 mg, about 0.6 mg, about 0.7 mg, about 0.8 mg, about 0.9 mg, about 1 mg, about 2 mg, about 2.5 mg, about 3 mg, about 3.5 mg, about 4 mg, about 4.5 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg per day.

In some embodiments, the combination can be, for example, 40 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 60 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 80 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 120 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 160 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 200 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 240 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 280 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 320 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 360 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 400 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 440 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily. In some embodiments, the combination can be, for example, 480 mg of 8-cyclopentyl-2-((4-(4-methylpiperazin-1 yl)phenyl)amino)-7-oxo-7,8-dihydropyrido[2,3-d]pyrimidine-6-carbonitrile and 2.5 mg of letrozole administered once daily.

In some embodiments, the combination is administered once daily. In some embodiments, the combination is administered once daily for 28 days (one cycle). In some embodiments, the combination is administered once daily in one or more 28 day cycles. In some embodiments, the combination is administered in a four-week cycle of consecutive once daily administration for three weeks, followed by one week with no administrations.

In some embodiments, the combination is administered once daily. In some embodiments, the combination is administered once daily for 28 days (one cycle). In some embodiments, the combination is administered once daily in one or more 28 day cycles. In some embodiments, the combination is administered in a four-week cycle of consecutive once daily administration for three weeks, followed by one week with no administrations.

In some embodiments, the present disclosure provides a method of treating hormone receptor positive endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating hormone receptor positive endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

In some embodiments, the present disclosure provides a method of treating progesterone receptor positive endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating progesterone receptor positive endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

In some embodiments, the present disclosure provides a method of treating estrogen receptor positive endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating estrogen receptor positive endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

In some embodiments, the present disclosure provides a method of treating recurrent metastatic low-grade endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating recurrent metastatic low-grade endometrial cancer in a subject in need thereof, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

In some embodiments, the present disclosure provides a method of treating a subject having a disease caused by a mutation, for example a POLE mutation, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating a subject having a disease caused by a mutation, for example a POLE mutation, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

In some embodiments, the present disclosure provides a method of treating a subject having a disease caused by a deficient mismatch repair (DMMR), the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating a subject having a disease caused by a deficient mismatch repair (DMMR), the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

In some embodiments, the present disclosure provides a method of treating a subject having a disease caused by a microsatellite instability (MSI), the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating a subject having a disease caused by a microsatellite instability (MSI), the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

In some embodiments, the present disclosure provides a method of treating a subject having a disease caused by altered p53 tumor suppression activity, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is once daily for at least 4 weeks.

In some embodiments, the present disclosure provides a method of treating a subject having a disease caused by altered p53 tumor suppressor activity, the method comprising administering to the subject a therapeutically-effective amount of the combination of 8-cyclopentyl-2-((4-(4-methylpiperazin-1 yl)phenyl)amino)-7-oxo-7,8-dihydropyrido[2,3-d]pyrimidine-6-carbonitrile monolactate and letrozole, wherein the administering is a 4-week cycle of: (i) a continuous, three-week period of once-daily administration; and ii) immediately following the three-week period, one week of no administration.

### EXAMPLES

### Example 1: Study to Evaluate an Oral Pharmaceutical Composition Disclosed Herein for Treating a Disease in a Subject

Summary: This study is a dose escalation study to investigate the safety, tolerability, and PK characteristics of 8-cyclopentyl-2-((4-(4-methylpiperazin-1-yl)phenyl)amino)-7-oxo-7,8-dihydropyrido[2,3-d]pyrimidine-6-carbonitrile (Compound 1) in patients with advanced cancers who have received and failed at least one prior treatment. The primary objective of this study is to assess the safety and tolerability of repeated daily dosing of compound 1 in patients with relapsed and/or refractory advanced cancers. The secondary objective of this study is to establish a maximum tolerated dose (MTD) and a recommend phase 2 dose (RP2D) of orally administered Compound 1. In addition, the study explores efficacy of Compound 1 in cancer patients.

Study Design: This study is a dose finding study using 3+3 design for dose escalation. Three to six patients are enrolled per dose cohort, followed by up to 12 additional patients at the RP2D. Approximately 36 patients with advanced cancers are enrolled in the study, based on 4 dose levels and an expansion cohort. If additional dose escalations are required to establish the MTD/RP2D, then 36 additional patients are added per dose level.

Compound (1) is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. The initial dose is 40 mg (one capsule) taken once daily for 28 days (one cycle). The dose is taken in the morning, on an empty stomach. Dose increments in the dose escalation 3+3 study are 40 mg of Compound 1 per cycle. Dose levels are 40 mg, 80 mg, 120 mg, 160 mg, etc., until a RP2D/MTD is reached. Each of the first three patients in the first and subsequent cohorts is assessed for dose limiting toxicities (DLT) during the first 28 days of treatment. If no patients experience a DLT, then enrollment to the next cohort begins at the next dose level. If one patient of the first three patients in a cohort experiences a DLT in the first 28 days, then an additional three patients are enrolled to that cohort for a total of six patients. If only one of the six patients in the cohort experiences a DLT in the first 28 days, then enrollment to the next cohort begins at the next dose level. If at any time two or more patients in a cohort experience a DLT in the first 28 days, the cohort is closed. The dose of that cohort is considered ‘non-tolerable’ and the prior dose level is defined as the MTD. Patients continue in the study until disease progression or intolerance or a decision to discontinue is reached.

### Study Objectives and Endpoints

The primary objective of this study is to assess the safety and tolerability of repeated daily dosing of Compound 1 in patients with relapsed and/or refractory advanced cancers. The primary endpoints include (DLTs, adverse events (AEs), deaths and other serious AEs.

The secondary objectives of this study are to establish a MTD of Compound 1 and a RP2D of orally administered Compound 1 and to characterize pharmacokinetics of Compound 1 following oral administration in patients with relapsed and/or refractory advanced cancers. Secondary endpoints include maximum plasma concentration (Cmax), area under the plasma concentration time curve (AUC), and half-life (t½).

Exploratory objectives of this study are to assess the efficacy of Compound 1, by objective responses per RECIST, wherever appropriate for applicable tumors. Assessment of non-Hodgkin's Lymphoma and CNS tumors is by imaging techniques (CT, PET, MRI).

### Pharmacokinetics

Blood samples are collected pre-and post-dose on days 1 and 8 of the first cycle and pre-dose on day 1 of cycles 2 and 3 for pharmacokinetic (PK) analysis. Compound (1) concentrations are determined in plasma samples by a validated liquid chromatography-tandem mass spectrometry (LC-MS/MS) assay. Levels of Compound 1 are determined at specified time points in the PK profile.

The following PK parameters are derived using model-independent analysis: time to reach Cmax (Tmax), Cmax, t½, AUC0-t, AUC0-α, CL, and Vss. Descriptive statistics (mean, median, range, standard deviation) for these parameters are provided and summarized by each dose group.

Cmax and Tmax are determined from the plasma concentration-time profile, and t½β is calculated as 0.693/k (where k is the terminal elimination rate constant, calculated by log-linear regression of the terminal portion of the concentration-time profile). AUC0-t is calculated by the linear trapezoidal rule and extrapolated to infinity using k to obtain AUC0-∞.

Pharmacokinetic parameters are calculated from Compound 1 concentration-time data using standard non-compartmental methods as implemented in WinNonlin. The maximum plasma concentration (Cmax) and time to reach Cmax (Tmax) are the observed values. The area under the plasma concentration-time curve (AUC) value is calculated to the last quantifiable sample (AUClast) by use of the linear trapezoidal rule. The AUC values are extrapolated to infinity (AUCinf) by dividing the last quantifiable concentration by the terminal disposition rate constant (λz), which is determined from the slope of the terminal phase of the concentration-time profile. The terminal half-life (T½) is calculated as 0.693 divided by λz. The apparent oral clearance (Cl/F) is calculated by dividing the dose administered by AUCinf.4. Pharmacokinetic data are analyzed by cohort.

Efficacy analysis. The efficacy variable is best overall response (ORR), using RECIST criteria, version 1.1. Objective tumor response is tabulated and summarized by the primary tumor type. If warranted, additional efficacy endpoints, like duration of response or time to progression, are analyzed.

### Example 2: Study to Evaluate PK/PD Results for a Pharmaceutical Composition Disclosed Herein for Treating a Disease in a Subject

Summary: This study is a dose escalation study to investigate the safety, tolerability, and PK characteristics of in-patients with advanced solid tumors who have received and failed at least one prior treatment. The primary objective of this study is to assess the safety and tolerability of repeated daily dosing of 8-cyclopentyl-2-((4-(4-methylpiperazin-1-yl)phenyl)amino)-7-oxo-7,8-dihydropyrido[2,3-d]pyrimidine-6-carbonitrile (Compound 1) in patients with relapsed and/or refractory advanced cancers. The secondary objective of this study is to establish a MT of Compound 1 and RP2D of orally administered Compound 1. In addition, the study explores efficacy of Compound 1 in cancer patients.

Study design: The study includes a treatment period (1 year) and a follow-up period (90 days after the last dose). Subjects are pathologically confirmed to have malignant solid tumors, or advanced (metastatic or unresectable) malignant solid tumors and have previously failed standard treatment (e.g., targeted therapy, chemotherapy, biotherapy, immunotherapy, etc.), as evidenced by disease progression or intolerance toxicity. Subjects can also be included in the study if there are currently no effective treatments for cancer.

The study is divided into two stages, including a dosage escalation and a dose expansion cohort. The first phase is a dose escalation, using 3+3 design to determine MTD and/or RP2D. Three to six patients are enrolled per dose cohort, followed by up to 12 additional patients at the RP2D. Approximately 9-30 patients are enrolled in the first phase. If additional dose escalations are required to establish the MTD/RP2D, then 36 additional patients are added per dose level.

Patients receive study drug orally under fasted conditions before breakfast. Compound (1) is administered once daily continuously for 3 weeks with one week break. Safety, tolerability, and dose-limiting toxicity are evaluated after 4 weeks (28 days) of dosing.

Compound (1) is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. The dose (one capsule) is taken in the morning, on an empty stomach. Dose increments in the dose escalation 3+3 study are 40 mg of Compound 1 per cycle. Dose levels are 40 mg, 80 mg, 120 mg, 160 mg, and 200 mg, or until a RP2D/MTD is reached. The highest escalation dose in the study is set at 200 mg. Dose escalation is performed as described in Example 1.

The second stage of the study is a dose expansion stage. The dose expansion stage enrolls 9-12 cancer patients (primarily advanced breast cancer and non-small cell lung cancer patients with HR(+) and HER2(−)). Test procedures are the same as in the dose expansion phase.

### Study Objectives and Endpoints

The primary objective of this study is to evaluate the tolerance, safety, and the anti-tumor efficacy of Compound 1 in patients having advanced solid tumors.

The secondary objective of this study is to characterize pharmacokinetics of Compound 1 following oral administration of single and multiple doses of Compound 1 in patients with relapsed and/or refractory advanced cancer. Secondary endpoints include maximum plasma concentration (Cmax), area under the plasma concentration time curve (AUC), and half-life (t½). The study evaluates the efficacy of Compound 1 in patients with solid tumors, including objective response rate ORR, progression-free survival PFS, duration of remission DOR, disease control rate DCR, etc.

### Pharmacokinetics

Blood samples are collected pre-and post-dose on days 1 and 8 of the first cycle and pre-dose on day 4 of the first cycle for pharmacokinetic (PK) analysis. Compound (1) PK is determined as described in Example 1.

Efficacy analysis. The efficacy variable of this study is best overall response (ORR), using RECIST criteria, version 1.1. Efficacy analysis includes: (1) Objective Remission Rate (ORR), defined as the proportion of subjects with complete remission (CR) and partial remission (PR) after treatment. (2) Disease Control Rate (DCR), defined as the proportion of subjects with complete remission (CR), partial remission (PR), and disease stabilization (SD) after treatment. (3) Time to remission (DOR), defined as the time from the initial recording of objective remission to the first occurrence of tumor progression, or death from any cause. And (4) Progression free survival (PFS), defined as treatment from initiation to tumor progression or death from any cause.

### Example 3: Dosing Regimen to Evaluate an Oral Pharmaceutical Composition Disclosed Herein With an Aromatase Inhibitor in a Subject

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with an aromatase inhibitor (e.g., letrozole) versus placebo in combination with an aromatase inhibitor (e.g., letrozole) for patients with estrogen receptor positive advanced or recurrent endometrial cancer.

Study Design: This study is a treatment response study using 1:1 randomized double-blind study. Patients are randomized into one of two treatment arms: Arm A: (placebo): letrozole-placebo combination therapy; and Arm B (experimental): letrozole-Compound 1 combination therapy. Each cycle in the study is 28 days of treatment with tumors assessed every 12 weeks.

Compound (1) is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound (1) is taken once daily for 28 days (one cycle). Alternatively, Compound 1 is taken once daily continuously for 3 weeks with one week of no administration for a total of 28 days. Treatment continues until progression of disease or unacceptable toxicity. Compound (1) is taken in the morning, on an empty stomach.

Letrozole is given in the form of a 2.5 mg tablet. One 2.5 mg letrozole tablet is taken once on days 1-28 (one cycle). Treatment continues until progression of disease or unacceptable toxicity. Letrozole is taken in the morning, at the same time as either Compound 1 or the placebo.

Primary outcome measures Primary outcome measures are increase in Progression-Free Survival (PFS) in experimental arm versus comparator arm.

Secondary Outcome Measures are:
1. PFS: increase in median PFS in experimental arm versus comparator arm.
2. Overall Response Rate (ORR) according to RECIST.
3. Disease Control Rate (DCR) for at least 12 weeks.
4. Time to First Subsequent Therapy (TFST): time from randomization to first subsequent therapy or death.
5. Progression-Free Survival 2 (PFS2): time from randomization to second objective disease progression or death.
6. Time to Second Subsequent Therapy (TSST): time from randomization to second subsequent therapy or death.
7. Patient Reported Outcomes (PROs) like Quality of Life questionnaire EORTC-QLQ-C30 & EORTC-QLQ-EN24. These are the validated questionnaires to be answered by patients. Results are reported as descriptive and on a scale of 1-10.
8. Number of participants with treatment-related adverse events as assessed by CTCAE v4.0.
9. Compliance in the two treatment arms.
10. Dose reductions/interruptions in the two treatment arms.

Toxicity/efficacy of the various compounds of the disclosure are analyzed and compared.

### Example 4: Dosing Regimen to Evaluate an Oral Pharmaceutical Composition Disclosed Herein With a Progestin in a Subject

The study is a randomized, double-blind, placebo-controlled, study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with a progestin (e.g., megestrol acetate) versus placebo in combination with a progestin (e.g., megestrol acetate) for patients with estrogen

Study Design: This study is a treatment response study using 1:1 randomized double-blind study. Patients are randomized into one of two treatment arms: Arm A: (placebo): megestrol acetate-placebo combination therapy; and Arm B (experimental): megestrol acetate—Compound 1 combination therapy. Each cycle in the study is 28 days of treatment with tumors assessed every 12 weeks.

Compound (1) is given in the form of a hard capsule comprising 48.4 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile. Compound (1) is taken once daily for 28 days (one cycle). Alternatively, Compound 1 is taken once daily continuously for 3 weeks with one week of no administration, for a total of 28 days. Treatment continues until progression of disease or unacceptable toxicity. Compound (1) is taken in the morning, on an empty stomach.

Megestrol acetate is given in the form of 625 mg of an oral suspension per day (5 mL of a 125 mg/mL suspension per day or one teaspoon daily). One 625 mg megestrol acetate dose is taken once on days 1-28 (one cycle). Treatment continues until progression of disease or unacceptable toxicity. Megestrol acetate is taken in the morning, at the same time as either Compound 1 or the placebo.

Primary outcome measures Primary outcome measures are increase in Progression-Free Survival (PFS) in experimental arm versus comparator arm.

Secondary Outcome Measures are:
1. PFS: increase in median PFS in experimental arm versus comparator arm.
2. Overall Response Rate (ORR) according to RECIST.
3. Disease Control Rate (DCR) for at least 12 weeks.
4. Time to First Subsequent Therapy (TFST): time from randomization to first subsequent therapy or death.
5. Progression-Free Survival 2 (PFS2): time from randomization to second objective disease progression or death.
6. Time to Second Subsequent Therapy (TSST): time from randomization to second subsequent therapy or death.
7. Patient Reported Outcomes (PROs) like Quality of Life questionnaire EORTC-QLQ-C30 & EORTC-QLQ-EN24. These outcomes are the validated questionnaires to be answered by patients. Results are reported as descriptive and on a scale of 1-10.
8. Number of participants with treatment-related adverse events as assessed by CTCAE v4.0.
9. Compliance in the two treatment arms.
10. Dose reductions/interruptions in the two treatment arms. Toxicity/efficacy of the various compounds of the disclosure are analyzed and compared.

### Example 5 In Vitro Multi-Kinase Activity-IC₅₀ Values

The study evaluates the IC₅₀ values (in nM) of Compound 1 in comparison to FDA approved CDK 4/6 inhibitors Comparator 1, Comparator 2 and Comparator 3, which represent treatment advances for HR+ HER²-metastatic breast cancer. IC₅₀ value is a quantitative measure indicating the concentration needed to inhibit the listed kinase by 50%. The results are shown in TABLE 1.

TABLE 1
IC₅₀ values (in nM)

| | | | |
|---|---|---|---|
| | Compar- | Compar- | Compar- |
| Compound 1 | ator 1 | ator 2 | ator 3 |

CDK Family

| | | | | |
|---|---|---|---|---|
| CDK4/cyclin D1 | 2 | 2 | 3 | 0.8 |
| CDK6/cyclin D1 | 0.6 | 0.8 | 2.0 | 0.6 |
| CDK1/cyclin B | 515 | >10,000 | >10,000 | 129 |
| CDK2/cyclin E | 69 | 2300 | >10,000 | 130 |
| CDK7/cyclin H | 3766 | >10,000 | >10,000 | 992 |
| CDK9/cyclinT1 | 48 | 630 | 390 | 7 |
| CDK16/cyclin Y | 25 | 2708 | >10,000 | 42 |
| CDK17/cyclin Y | 14 | 2528 | >10,000 | 29 |

Other Kinases

| | | | | |
|---|---|---|---|---|
| CSF1R | 0.7 | >10,000 | >10,000 | >10,000 |
| ARK 5/NUAK 1 | 5 | 1,400 | 1,540 | 773 |
| KIT | 8.5 | >10,000 | >10,000 | 7368 |

TABLE 1 shows that Compound 1 had comparable IC₅₀ values for CDK4 and CDK6 as Comparator 1, Comparator 2, and Comparator 3. Compound 1 had significantly lower IC₅₀ values (1 to 3 order of magnitude lower) for CSF1R, ARK 5, and KIT, than Comparator 1, Comparator 2, and Comparator 3. Compound 1 had also significantly lower IC₅₀ values (1 to 3 order of magnitude lower) for CDK 1, CDK 2, CDK 7, CDK 9, CDK 16, and CDK 17, than Comparator 1 and Comparator 2. This study demonstrates that Compound 1 has a high efficacy for inhibiting a number of kinases.

### Example 6 Dosing Regimen to Evaluate an Oral Pharmaceutical Composition Disclosed Herein in a Subject

The study is a phase 1 dose-escalation study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in patients with advanced cancer. Patients are dosed daily in 28-day cycles with Compound 1 starting at 40 mg daily with dosages escalating 40 mg/day increments for each new cohort or only on days 1-21 of 28-day cycles. No dose-limiting toxicities are observed with the 200 mg/day cohort. Adverse events reported are Grade 1 or 2 in severity and are predominantly gastrointestinal (GI) disorders.

### Example 7 Dosing Regimen to Evaluate an Oral Pharmaceutical Composition Disclosed Herein in Combination With an Aromatase Inhibitor in a Subject

The study is an open-label multicenter study of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with an aromatase inhibitor (e.g., letrozole) for patients with endometrial cancer.

Study Design: This study is a Phase 1/2a, open label, multi-center study to evaluate safety and efficacy of escalating doses of 8-cyclopentyl-2-((4-(4-methylpiperazin-1-yl)phenyl)amino)-7-oxo-7,8-dihydropyrido[2,3-d]pyrimidine-6-carbonitrile (Compound 1) in combination with letrozole orally once daily. Approximately 30 patients with endometrial cancer are enrolled in the Phase 1 study. The primary objective of the phase 1 study is to investigate the safety, tolerability, pharmacokinetics (PK), and pharmacodynamics (PD) of escalating doses of Compound 1 administered orally, once daily, in combination with letrozole 2.5 mg orally, once daily, and to determine the recommended phase 2 dose (RP2D). Dose escalation follows a standard 3+3 design. Approximately 20 patients with endometrial cancer are enrolled in the phase 2a study. Treatment will continue until disease progression, patient withdrawal, or unacceptable drug-related toxicity. The primary objective of the phase 2a study is to investigate the efficacy of Compound 1 administered orally, once daily, in combination with letrozole 2.5 mg orally, once daily, measured by the metric of progression free survival (PFS) at 24 weeks determined by investigator assessment.

Compound 1 is given in the form of a clear, hard, capsule comprising 40 mg or 60 mg 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile as 8-cyclopentyl-2-((4-(4-methylpiperazin-1-yl)phenyl)amino)-7-oxo-7,8-dihydropyrido[2,3-d]pyrimidine-6-carbonitrilemonolactate salt, equivalent to 40 mg of 8-cyclopentyl-2-((4-(4-methylpiperazin-1-yl)phenyl)amino)-7-oxo-7,8-dihydropyrido[2,3-d]pyrimidine-6-carbonitrile monolactate. The starting dose of Compound 1 is 200 mg taken once daily for a treatment cycle of 28 days (one cycle) in a 3+3 design. Following one treatment cycle, the dose of Compound 1 will escalate with 40 mg/day increments with a new cohort enrolled at the increased dosage (e.g. 240 mg taken once daily for a treatment cycle) and the previous cohort allowed to escalate to the escalated dose. Treatment continues until the minimally effective biological dose (MBED) or the maximum tolerated dose (MTD) is reached and the RP2D is determined. In the phase 2a study, Compound 1 is given at the RP2D from phase 1. Compound 1 is taken in the morning, on an empty stomach with water at least one hour before starting breakfast.

Compound 1 dose modifications and management guidelines are dependent on the adverse event of special interest (AESI). Dose reduction is not permitted for the first 28 days in any of the dose-escalation cohorts but is permitted thereafter. Any dose modification requirement below 200 mg/day Compound 1 will result in withdrawal from the study. Dose escalation occurs after the cohort has completed 28 days of dosing with no dose-limiting toxicity observed (n=3) or with no more than 1 patient experiencing a dose-limiting toxicity (n=6).

Dose modification for neutropenia: Complete blood counts are monitored prior to the start of Compound 1 administration and as clinically indicated. Dose modification is dependent on the Common Terminology Criteria for Adverse Events (CTCAE Grade):
i. CTCAE Grade 1 or 2: no dose modification of Compound 1 required
ii. CTCAE Grade 3: suspend dose until toxicity resolves to Grade 2 or less, dose reduction not required
iii. CTCAE Grade 3 recurrent: suspend dose until toxicity resolves to Grade 2 or less, resume at next lower dose
iv. CTCAE Grade 4: discontinue Compound 1

Letrozole is given in the form of a 2.5 mg tablet. One 2.5 mg letrozole tablet is taken once on days 1-28 (one cycle). Treatment continues until progression of disease or unacceptable toxicity. Letrozole is taken in the morning, at the same time as Compound 1.

Phase 1 primary outcome measures: Primary outcome measures are safety, tolerability, PK, and PD of escalating doses of Compound 1 administered orally, once daily, in combination with letrozole 2.5 mg orally, once daily.

Phase 2a primary outcome measures: Primary outcome measures are efficacy of Compound 1 administered orally, once daily, at the RP2D in combination with letrozole 2.5 mg orally, once daily measured by PFS at 24 weeks.

Phase 1 Secondary Outcome Measures are the MBED and/or MTD and RP2D of Compound 1 administered orally, once daily, in combination with letrozole 2.5 mg orally, once daily.

Phase 1 and 2a Secondary Outcome Measures are the following efficacy endpoints at 16 and at 24 weeks, as assessed by using the Response Evaluation Criteria in Solid Tumors (RECIST) version 1.1:
1. PFS at 16 weeks by investigator assessment
2. Median PFS by investigator assessment
3. Complete response (CR) rate
4. Partial response (PR) rate
5. Stable disease (SD) rate
6. Overall Response Rate (ORR equals CR+PR)
7. Disease Control Rate (DCR equals CR+PR+SD)
8. Duration of response (DoR)
9. Time to response (TTR)
10. Overall survival (OS)
   Additionally, the secondary outcome measure of the phase 1 and phase 2a trials is to evaluate the safety and tolerability of the RP2D of Compound 1 in combination with letrozole 2.5 mg orally, once daily.

Phase 1 and phase 2a exploratory measures are:
1. Estrogen/progesterone receptor status
2. RB status
3. CCNE1/CCND1 expression in tissue
4. CCNE1/CCND1 expression in ctDNA
5. POLE molecular classification
6. DMMR molecular classification
7. MSI molecular classification
8. P53 molecular classification
9. PK parameters, including:
   i. Maximum plasma concentration of drug [Cₘₐₓ]
   ii. Time to reach Cₘₐₓ (Tₘₐₓ)
   iii. Terminal half-life [T_{1/2}]
   iv. Area under the concentration-time curve [AUC] from time 0 to time of last quantifiable sample [AUC₀₋ₜ]
   v. AUC extrapolated to infinity AUC_{0-∞}],
   vi. Clearance [CL]
   vii. Volume of distribution at steady state [Vss]
10. PD assessments.

Pharmacokinetics measurements: Pharmacokinetic (PK) samples will be collected from all patients on Days 1 and 8 of first 28-day cycle. PK samples will be drawn predose and then 0.5, 1, 2, 4, 6, 8, 10, and 24 hours post dose on both days. 24-hour samples will be collected on Days 2 and 9 of the 28-day cycle prior to dosing and a single predose PK sample will be drawn on Day 1 of Cycles 2 and 3.

Pharmacodynamic assessments: Pharmacodynamic (PD) samples for serum TK1 activity levels will be drawn at screening, predose on Days 1, 8, 15, 22 at dose Cycle 1, and then at predose Day 1 of every dose cycle and at end of trial (EOT). The effect of Compound 1 letrozole combination treatment is assessed using the validated DiviTum'TKa technology. PD parameters are calculated based on changes in serum TKa levels using pretreatment and on-treatment serial measurements with relationships between TKa, drug PK, and patient response to Compound 1 letrozole combination treatment analyzed with respect to each timepoint, each dosing cohort and each specific diagnosis/tumor type. Descriptive statistics for each parameter are provided and summarized by each dose group.

Inclusion criteria patients meet the following criteria for inclusion in the study:
1. Are 18 years of age, or are the legal age of consent in jurisdiction of study, at time of signing of informed consent form
2. Have recurrent metastatic disease or advanced (Stage IV) disease
3. Have received 1 or 2 prior lines of systemic therapy for metastatic disease
4. Have not received more than 2 prior lines of system therapy for endometrial cancer, including checkpoint inhibitor, hormone therapy, or chemotherapy. Prior external beam radiotherapy, brachytherapy, and/or surgery for localized disease is allowed and not counted as a line of therapy
5. Have measurable disease outside the radiated field
6. Have local mismatch repair (MMR) immunohistochemistry results available; dMMR and

MMRp patients are eligible and will be documents for research purposes
7. Have Eastern Cooperative Oncology Group Performance Status (ECOG PS) of 0 or 1
8. Have tissue available for estrogen/progesterone receptor status and molecular classification analysis
9. Have adequate organ function as indicated by the following:
   a. Absolute neutrophil count (ANC) ≥1.0×10⁹/L
   b. Platelets ≥100×10⁹/L
   c. Hemoglobin ≥9.0 g/dL
   d. International Normalized Ratio (INR) ≤1.5
   e. Creatinine clearance ≥60/mL, as estimated by Cockcroft-Gault equation
   f. Alanine aminotransferase (ALT) and aspartate aminotransferase (AST) below 3.0×the upper limit of normal (ULN) (or ALT and AST ≤5×ULN if liver metastases are present).
   g. Total serum bilirubin <1.5×ULN; or total bilirubin ≤3.0×ULN with direct bilirubin within normal range of the central laboratory in participants with well documented Gilbert's Syndrome.
10. Have baseline QTc interval <470 msec.
11. Is able to swallow oral medications.
12. Have a life expectancy of at least 12 weeks
13. Sex and Contraceptive/Barrier Requirements
   a. Is postmenopausal, defined as:
      i. Patient's last menstrual period occurred more than 12 months prior to screening without any alternative medical cause, and
      ii. Patient's postmenopausal status is confirmed by screening serum follicle-stimulating hormone concentration of >40 mIU/mL; or
      iii. Patient has undergone surgical sterilization (bilateral oophorectomy and/or hysterectomy)
   OR
   b. Must have a negative pregnancy test at screening and upon study entry (Cycle 1 Day 1) if not postmenopausal and
   c. Contraceptive use must be consistent with local regulations regarding the methods of contraception for those participating in clinical studies if not postmenopausal. Patients under 55 years with intact ovaries will undergo hormonal verification.
14. Is capable of giving signed informed consent, which includes compliance with the requirements and restrictions listed in the ICF and in this protocol.

Exclusion criteria: Patients are excluded in the study if they exhibit any of the following:
1. Have received a CDK 4/6 inhibitor in the past.
2. Have any significant medical condition, laboratory abnormality, or psychiatric illness that, in the opinion of the Investigator, would prevent the patient from participating in the study or present an unacceptable risk to the patient.
3. Are at risk for Torsades de pointes (TdP): Patients who have a marked baseline prolongation of QT/QTc interval (eg, repeated demonstration of a QTc interval >470 msec) using Fredericia's QT correction formula, or who have a history of additional risk factors for TdP (eg, heart failure, hypokalemia, family history of Long QT Syndrome), or who are currently taking medications that prolong the QT/QTc interval.
4. Have uncontrolled intercurrent or significant medical illness serious underlying medical condition, abnormal laboratory finding, or psychiatric illness/social situation that might, in the Investigator's or the Sponsor's judgment, prevent the participant from receiving study treatment or being followed in this study, or otherwise renders the participant inappropriate for the study, including but not limited to ongoing or active infection, bleeding, congestive heart failure, unstable angina, cardiac arrhythmia, oxygen-dependent lung disease, and psychiatric illness/social situations that limit participation compliance with study procedures and requirements.
5. Are currently taking or within 5 half-lives of taking strong inducers and inhibitors of cytochrome P450 enzyme (CYP)2C8 and CYP3A4.
6. Have a recent history of venous thromboembolic events, defined as event occurring <6 months prior to screening and also currently on therapy, known underlying hypercoagulability, or a major thromboembolic event within the past 2 years.
7. Have baseline Grade ≥2 diarrhea.
8. Have Grade ≥3 hypercalcemia (corrected serum calcium >12.5 mg/dL).
9. Are pregnant or nursing mothers.
10. Have had major surgery within 14 days prior to screening to allow for postoperative healing of the surgical wound and site(s).
11. Have received recent (within 28 days prior to screening) live attenuated vaccines.
12. Have active infection, including bacterial or fungal infections or active viral infection or viral load, including any human immunodeficiency virus (HIV), or hepatitis B (HBV) virus, hepatitis C virus (HCV), or SARS-CoV-2 (COVID-19).
13. Currently has or has been treated in the past 2 years, for any other cancer or malignancy, except:
   a. Non-melanoma skin cancer, including basal cell carcinoma of the skin
   b. Curatively treated carcinoma in situ of the cervix.
14. Have any clinically significant, uncontrolled heart disease, and/or cardiac repolarization abnormality. Has a history of any of the following:
   a. Syncope of cardiovascular etiology
   b. Ventricular arrhythmia of pathological origin
   c. Sudden cardiac arrest
   d. Documented history of congestive heart failure with reduced ejection fraction.
15. Have interstitial pneumonia or has severe impairment of lung function defined as:
   a. Vital capacity and diffusing capacity of the lung for carbon monoxide (DLCO) of ≤50% of the normal predicted values, or
   b. Oxygen (O2) saturation at rest in ambient environment of ≤88%.
16. Has received within the 21 days prior to screening, is currently receiving, or intends to receive during the study any nonstudy anticancer therapy, including but not limited to any of the following:
   a. Anticancer agent
   b. Investigational agent
   c. Surgical intervention
   d. Radiation intervention, including any radiation therapy (includes radiation to an isolated lesion). (Palliative radiation to lesions that are not target lesions is permissible).
17. Have central nervous system metastases or leptomeningeal carcinomatosis.
18. Is not a candidate for treatment with letrozole

Phase 1 and phase 2a schedule of assessments: The phase 1 and phase 2a trials proceed according to the schedule:
i. Informed consent is obtained from −28 days to −1 day prior to onset of first dose cycle
ii. Participant history and study eligibility review
   a. Demography at −28 days to −1 day prior to onset of first dose cycle
   b. Inclusion/exclusion criteria at −28 days to −1 day and-7 to −1 day prior to onset of first dose cycle and on day 1 the first dose cycle
   c. Medical history and current medical conditions at −28 days to −1 day prior to onset of first dose cycle
   d. Tumor histology at −28 days to −1 day prior to onset of first dose cycle
   e. Diagnosis and extent of cancer at −28 days to −1 day prior to onset of first dose cycle
   f. Prior antineoplastic therapy at −28 days to −1 day prior to onset of first dose cycle
   g. Prior and concomitant medications beginning at −28 days to −1 day and throughout the duration of the treatment cycle until the end of trial or end of dosing, within 15 days after last dose
iii. Physical examinations
   a. Physical examination comprising a full physical examination beginning at −7 days to −1 day prior to onset of first dose cycle and at Day 1 of each subsequent dose cycle and at end of trial or end of dosing, within 15 days after last dose
   b. ECOG PS beginning at −7 days to −1 day prior to onset of first dose cycle, at day 1 of first dose cycle, at day 1 of each subsequent dose cycle, and at end of trial or end of dosing, within 15 days after last dose.
   c. Height at −7 days to −1 day prior to onset of first dose cycle
   d. Body weight at −7 days to −1 day prior to onset of first dose cycle, at day 1 of first dose cycle, at day 1 of each subsequent dose cycle, and at end of trial or end of dosing, within 15 days after last dose
   e. Vital signs at −7 days to −1 day prior to onset of first dose cycle, at days 1 and 15 of first dose cycle, at day 1 of each subsequent dose cycle, and at end of trial or end of dosing, within 15 days after last dose.
iv. Laboratory assessments
   a. Hematology at −7 days to −1 day prior to onset of first dose cycle, at days 8±2 and day 15 of the first dose cycle, at day 1 of each subsequent dose cycle, and at end of trial or end of dosing, within 15 days after last dose
   b. Chemistry at −7 days to −1 day prior to onset of first dose cycle, at days 8±2 and day 15 of the first dose cycle, at day 1 of each subsequent dose cycle, and at end of trial or end of dosing, within 15 days after last dose
   c. Fasting lipid panel at −7 days to −1 day prior to onset of first dose cycle and at end of trial or end of dosing, within 15 days after last dose
   d. Fasting glucose at −7 days to −1 day prior to onset of first dose cycle and at end of trial or end of dosing, within 15 days after last dose
   e. Coagulation at −7 days to −1 day prior to onset of first dose cycle and at end of trial or end of dosing, within 15 days after last dose
   f. Urinalysis at −7 days to −1 day prior to onset of first dose cycle, at day 1 of dose cycle 2, at day 1 of dose cycle 3, and at end of trial or end of dosing, within 15 days after last dose
   g. Pregnancy test at −7 days to −1 day prior to onset of first dose cycle, at day 1 of first dose cycle, at day 1 of each subsequent dose cycle, and at end of trial or end of dosing, within 15 days after last dose
v. Pharmacokinetic and Pharmacodynamic assessments
   a. Blood collection for PK sampling at day 1 of first dose cycle, at day 8±2 of first dose cycle, at day 1 of second dose cycle, at day of third dose cycle
   b. Blood collection for PD sampling at −7 days to −1 day prior to onset of first dose cycle; at days 1, 8±2, 15, and 22 of first dose cycle, at day 1 of each subsequent dose cycle, and at end of trial or end of dosing, within 15 days after last dose
vi. Tumor Assessments
   a. Tumor evaluation (per RECIST v1.1) at −28 days to −1 day prior to onset of first dose cycle and at every 8 weeks during the first year of study and then every 12 weeks thereafter until disease progression, death, withdrawal of consent, loss to follow up, or participant/guardian decision, and at end of trial or end of dosing. If PR/CR is reported confirmatory assessment is performed at last 4 weeks after response is first documents
vii. Cardiac Assessments
   a. 12-lead ECG at −7 days to −1 days prior to onset of first dose cycle, at days 1 and 8±2 of first dose cycle, at day 1 of each subsequent dose cycle, up to 5 cycles, and at end of trial or end of dosing, within 15 days after last dose.
   b. ECHO or MUGA with EF during treatment phase as clinically indicated per investigator
viii. Other safety assessments
   a. Adverse events and Serious Adverse Event review continuous through 30 days after last dose and 30 days after last dose
ix. Study Intervention administration
   a. Compound (1) days 1 to 28 of each dose cycle
   b. Letrozole (2.5 mg orally, daily) days 1 to 28 of each dose cycle
x. Follow-up assessments
   a. Collection of information on new anticancer therapies, as applicable at end of trial or end of dosing, within 15 days after last dose; during safety follow-up 30 days after last dose, and at survival follow-up every 12 weeks
   b. PFS and survival follow up at 30 days after last dose and every 12 weeks after end of trial.

### Embodiments

Embodiment 1. A method of treating hormone receptor positive endometrial cancer in a subject in need thereof, the method comprising administering to the subject a therapeutically-effective amount of a compound of formula (I)

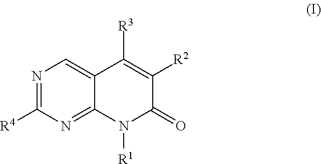

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
provided that, when R⁴ is —NR⁵R⁶ and one of R⁵ and R⁶ is pyridyl, then R² is not —C(O)CH₃,
or a pharmaceutically-acceptable salt thereof.

Embodiment 2. A method of treating endometrial cancer in a subject in need thereof, the method comprising administering to the subject a therapeutically-effective amount of a compound of formula (I)

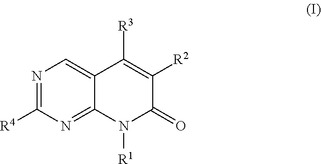

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
provided that, when R⁴ is —NR⁵R⁶ and one of R⁵ and R⁶ is pyridyl, then R² is not —C(O)CH₃,
or a pharmaceutically-acceptable salt thereof;
wherein the subject received a therapy other than the compound for the endometrial cancer prior to the administering.

Embodiment 3. A method of treating endometrial cancer in a subject in need thereof, the method comprising administering to the subject a therapeutically-effective amount of a compound of formula (I)

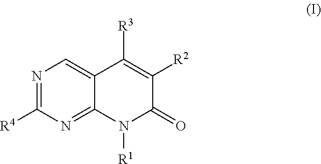

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
provided that, when R⁴ is —NR⁵R⁶ and one of R⁵ and R⁶ is pyridyl, then R² is not —C(O)CH₃,
or a pharmaceutically-acceptable salt thereof;
wherein the administering is once daily for at least 4 weeks.

Embodiment 4. A method of treating endometrial cancer in a subject in need thereof, the method comprising administering to the subject a therapeutically-effective amount of a compound of formula (I)

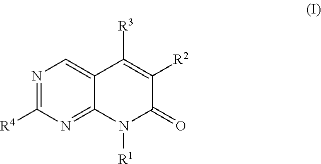

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
provided that, when R⁴ is —NR⁵R⁶ and one of R⁵ and R⁶ is pyridyl, then R² is not —C(O)CH₃,
or a pharmaceutically-acceptable salt thereof;
wherein the administering is a 4-week cycle of:
   (i) a continuous, three-week period of once-daily administration; and
   ii) immediately following the three-week period, one week of no administration.

Embodiment 5. A method of treating endometrial cancer in a subject in need thereof, the method comprising:
(i) administering to the subject a therapeutically-effective amount of a first compound, wherein the first compound is a compound of formula (I):

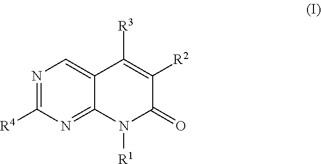

wherein:
   R¹ is alkyl, alkenyl, alkynyl, cycloalkyl, aryl, heteroaryl or heterocyclyl, each of which is unsubstituted or substituted, or hydrogen;
   R² is alkyl, alkenyl, alkynyl, cycloalkyl, aryl, heteroaryl, heterocyclyl, —CN, —C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen;
   R³ is alkyl, alkenyl, alkynyl, cycloalkyl, aryl, heterocyclyl or heteroaryl, each of which is unsubstituted or substituted, or hydrogen or halogen;
   R⁴ is alkyl, alkenyl, alkynyl, cycloalkyl, aryl, heterocyclyl, heteroaryl, —CN, C(O)R⁵, —C(O)OR⁵, —C(O)NR⁵R⁶, —C(═N)NR⁵R⁶, —OR⁵, —SR⁵, —NR⁵R⁶, —NR⁵C(O)R⁶, —NR⁵C(O)OR⁶, —OC(O)R⁵, —OC(O)NR⁵R⁶, —S(O)₂R⁵, —NHS(O)₂R⁵, or —OS(O)₂R⁵, each of which is independently substituted or unsubstituted, or hydrogen or halogen; and
   each R⁵ and R⁶ is independently alkyl, alkenyl, alkynyl, cycloalkyl, aryl, heterocyclyl, or heteroaryl, each of which is independently substituted or unsubstituted, or hydrogen or halogen,
provided that, when R⁴ is —NR⁵R⁶ and one of R⁵ and R⁶ is pyridyl, then R² is not —C(O)CH₃,
or a pharmaceutically-acceptable salt thereof; and
   (ii) administering to the subject a therapeutically-effective amount of a second compound.

Embodiment 6. A method of treating hormone receptor positive endometrial cancer in a subject in need thereof, the method comprising orally administering to the subject a solid pharmaceutical composition, the solid pharmaceutical composition comprising 40 mg to 500 mg of a compound that is 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the endometrial cancer prior to the administering, wherein the therapy was received after the subject was diagnosed with endometrial cancer, and wherein the subject has not responded to the therapy prior to the administering; and
wherein the administering comprises 3 weeks of once-daily administration.

Embodiment 7. A method of treating endometrial cancer in a subject in need thereof, the method comprising:
(i) orally administering to the subject a solid pharmaceutical composition comprising 40 mg to 500 mg 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the endometrial cancer prior to the administering, wherein the therapy was received after the subject was diagnosed with endometrial cancer, and wherein the subject has not responded to the therapy prior to the administering; and
wherein the administering comprises 3 weeks of once-daily administration; and
(ii) orally administering to the subject a therapeutically-effective amount of letrozole.

Embodiment 8. A method of treating endometrial cancer in a subject in need thereof, the method comprising:
(i) orally administering to the subject a solid pharmaceutical composition comprising 40 mg to 500 mg 8-cyclopentyl-2-((4-(4-methylpiperazin-1-yl)phenyl)amino)-7-oxo-7,8-dihydropyrido[2,3-d]pyrimidine-6-carbonitrile monolactate,
wherein the subject received a therapy other than the compound for the endometrial cancer prior to the administering, wherein the therapy was received after the subject was diagnosed with endometrial cancer, wherein the subject has not responded to the therapy prior to the administering; and
wherein the administering comprises 3 weeks of once-daily administration; and
(ii) orally administering to the subject a therapeutically-effective amount of megestrol acetate.

1.-343. (canceled)

344. A method of treating hormone receptor positive endometrial cancer in a subject in need thereof, the method comprising administering to the subject a therapeutically-effective amount of a compound, wherein the compound is 8-cyclopentyl-2-((4-(4-methylpiperazin-1-yl)phenyl)amino)-7-oxo-7,8-dihydropyrido[2,3-d]pyrimidine-6-carbonitrile or a pharmaceutically-acceptable salt thereof.

345. The method of claim 344, wherein the compound is 8-cyclopentyl-2-((4-(4-methylpiperazin-1-yl)phenyl)amino)-7-oxo-7,8-dihydropyrido[2,3-d]pyrimidine-6-carbonitrile monolactate.

346. The method of claim 344, wherein the therapeutically-effective amount is about 40 mg to about 500 mg per day.

347. The method of claim 344, wherein the therapeutically-effective amount is about 40 mg to about 280 mg per day.

348. The method of claim 344, wherein the hormone receptor positive endometrial cancer is an estrogen receptor positive endometrial cancer.

349. The method of claim 344, wherein the administering is oral.

350. The method of claim 344, wherein the administering is intravenous.

351. The method of claim 344, further comprising administering to the subject a therapeutically-effective amount of a second compound.

352. The method of claim 351, wherein the second compound is an estrogen receptor modulator.

353. The method of claim 351, wherein the second compound is an estrogen receptor blocker.

354. The method of claim 351, wherein the second compound is an aromatase inhibitor.

355. The method of claim 351, wherein the second compound is letrozole or a pharmaceutically-acceptable salt thereof.

356. The method of claim 355, wherein the therapeutically-effective amount of the second compound is about 2.5 mg of letrozole.

357. The method of claim 351, wherein the second compound is a progestin.

358. The method of claim 357, wherein the progestin is megestrol acetate.

359. The method of claim 351, wherein the administering of the second compound is once daily.

360. The method of claim 351, wherein the administering of the second compound is at least once daily.

361. The method of claim 351, wherein the second compound is a selective estrogen receptor degrader.

362. The method of claim 351, wherein the second compound is a vascular endothelial growth factor inhibitor.

363. The method of claim 351, wherein the second compound is a phosphoinositide 3-kinase inhibitor.
